SEQUENCE LISTING (45 sequences)

SEQ ID NO: 1 - protein, 14 aa, Synthetic construct
TRGVEEVAEGVVLL

SEQ ID NO: 2 - protein, 17 aa, Synthetic construct
SGAGSGGSGTGSGSGGS

SEQ ID NO: 3 - protein, 6 aa, Synthetic construct
RYSPNL

SEQ ID NO: 4 - protein, 18 aa, Synthetic construct
ARGTYRSERPROASNLEU

SEQ ID NO: 5 - protein, 7 aa, Synthetic construct
ENLYFQM

SEQ ID NO: 6 - protein, 7 aa, Synthetic construct
PKKKRKV

SEQ ID NO: 7 - protein, 12 aa, Synthetic construct
LQLPPLERLTLD

SEQ ID NO: 8 - protein, 10 aa, Synthetic construct
LALKLAGLDI

SEQ ID NO: 9 - DNA, 45 bp, Synthetic construct
  source 1..45: mol_type = other DNA
ggctctggtgctagcgactacaaggatgacgacgataagactagt

SEQ ID NO: 10 - protein, 86 aa, Synthetic construct
FDAVTDIIIKENLKDCGLFGGATCCACCCTGGAAGTAGAGATTTTCMNNMNNMNNMNNMNNMNNCGCCACTTCCTCCACTCCACGC

SEQ ID NO: 11 - protein, 70 aa, Synthetic construct
GGATCCACCCTGGAAGTAGAGATTTTCMNNMNNMNNMNNMNNMNNMNNCGCCACTTCCTCCACTCCACGC

SEQ ID NO: 12 - protein, 73 aa, Synthetic construct
GGATCCACCCTGGAAGTAGAGATTTTCMNNMNNMNNMNNMNNMNNMNNMNNCGCCACTTCCTCCACTCCACGC

SEQ ID NO: 13 - protein, 76 aa, Synthetic construct
GGATCCACCCTGGAAGTAGAGATTTTCMNNMNNMNNMNNMNNMNNMNNMNNMNNCGCCACTTCCTCCACTCCACGC

SEQ ID NO: 14 - DNA, 45 bp, Synthetic construct; identical to SEQ ID NO: 9
  source 1..45: mol_type = other DNA

SEQ ID NO: 15 - DNA, 66 bp, Synthetic construct
  source 1..66: mol_type = other DNA
ctcgagctattaagcgtaatctggaacgtcatatgggtaggatccaccctggaagtagagattttc

SEQ ID NO: 16 - protein, 79 aa, Synthetic construct
GCTAGCGCAGCGAATGATGAAAATTACTTCNNKNNKNNKCCTAATTTGNNKNNKNNKGGATCAGGCGGTTCTGGTACTG

SEQ ID NO: 17 - protein, 77 aa, Synthetic construct
GCTAGCGCAGCGAATGATGAAAATTACTTCNNKNNKCCTAATTTGNNKNNKNNKNNKTCAGGCGGTTCTGGTACTGG

SEQ ID NO: 18 - DNA, 19 bp, Synthetic construct
  source 1..19: mol_type = other DNA
ctgcagcaaggtctgcagg

SEQ ID NO: 19 - DNA, 30 bp, Synthetic construct
  source 1..30: mol_type = other DNA
tcagatctcgagctattacttatcgtcgtc

SEQ ID NO: 20 - DNA, 30 bp, Synthetic construct; identical to SEQ ID NO: 19
  source 1..30: mol_type = other DNA

SEQ ID NO: 21 - protein, 61 aa, Synthetic construct
GAGATGGTTTCCACCTGCACTCCMNNMNNMNNMNNMNNMNNTCCAGTACCAGAACCGCCTG

SEQ ID NO: 22 - DNA, 72 bp, Synthetic construct
  source 1..72: mol_type = other DNA
ctgcagcaaggtctgcaggctagtggtggaggaggctctggtgctagcgcagcgaatgatgaaaattacttc

SEQ ID NO: 23 - DNA, 72 bp, Synthetic construct; identical to SEQ ID NO: 22
  source 1..72: mol_type = other DNA

SEQ ID NO: 24 - DNA, 23 bp, Synthetic construct
  source 1..23: mol_type = other DNA
ggagtgcaggtggaaaccatctc

SEQ ID NO: 25 - DNA, 30 bp, Synthetic construct; identical to SEQ ID NO: 19
  source 1..30: mol_type = other DNA

SEQ ID NO: 26 - DNA, 771 bp, Synthetic construct
  source 1..771: mol_type = other DNA
ggcgtccaagtcgagacaatatctcccggcgacggacgaacgtttccgaagcggggacagacttgcgtcgttcactacacaggaatgctggaggatggcaaaaaggtcgattccagccgggacaggaataagccgttcaaattcatgctgggaaaacaggaggtaatacgaggttgggaggagggtgtggcgcaaatgtctgtcggtcagcgggcgaaactgaccatttctcctgattatgcttacggagcgacaggccatccggggatcattccgccccacgctaccctggtattcgatgtagaactcttgaagctcgaaaccagaggggttgaagaagtagccagatattcccccaacctggcagcgaatgatgaaaattacggtactcctaatttgcggccttttggttcaggcggttctggtactggatctggttctggaggttctggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 27 - protein, 16 aa, Synthetic construct
MKTIIALSYIFCLVFA

SEQ ID NO: 28 - protein, 5 aa, Synthetic construct
YGGFM

SEQ ID NO: 29 - protein, 127 aa, Synthetic construct
PNLRPFGSGGSGTGSGSGGSGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 30 - skipped

SEQ ID NO: 31 - protein, 15 aa, Synthetic construct
GGSGSGSGGSGGSGG

SEQ ID NO: 32 - protein, 41 aa, Synthetic construct
LPTWSTPVQPMALIVLGGVAGLLLFIGLGIFFCVRCRHRRR

SEQ ID NO: 33 - protein, 23 aa, Synthetic construct
KGSGSTSGSGSGGSRGSGGSSGG

SEQ ID NO: 34 - protein, 171 aa, Synthetic construct
MNGAIGGDLLLNFPDMSVLERQRAHLKYLNPTFDSPLAGFFADSSMITGGEMDSYLSTAGLNLPMMYGETTVEGDSRLSISPETTLGTGNFKAAKFDTETKDCNEAAKKMTMNRDDLVEEGEEEKSKITEQNNGSTKSIKKMKHKAKKEENNFSNDSSKVTKELEKTDYIH

SEQ ID NO: 35 - protein, 9 aa, Synthetic construct
YPYDVPDYA

SEQ ID NO: 36 - DNA, 48 bp, Synthetic construct
  source 1..48: mol_type = other DNA
atgaagaccatcatcgccctgagctacatcttctgcctggtgttcgcc

SEQ ID NO: 37 - DNA, 15 bp, Synthetic construct
  source 1..15: mol_type = other DNA
tacggcggatttatg

SEQ ID NO: 38 - DNA, 381 bp, Synthetic construct
  source 1..381: mol_type = other DNA
cctaatttgcggccttttggttcaggcggttctggtactggatctggttctggaggttctggagtgcaggtggaaaccatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 39 - skipped

SEQ ID NO: 40 - DNA, 45 bp, Synthetic construct
  source 1..45: mol_type = other DNA
ggtggaagtggatcaggcagcggtggatctggaggtagcggcgga

SEQ ID NO: 41 - DNA, 123 bp, Synthetic construct
  source 1..123: mol_type = other DNA
ctgcccacatggtccaccccggtgcagccaatggccctgattgtgctggggggcgtcgccggcctcctgcttttcattgggctaggcatcttcttctgtgtcaggtgccggcaccgaaggcgc

SEQ ID NO: 42 - DNA, 69 bp, Synthetic construct
  source 1..69: mol_type = other DNA
aagggctcgggctcgacctcgggctcgggcagcggtggctctagaggttctggtggcagctctggaggt

SEQ ID NO: 43 - DNA, 513 bp, Synthetic construct
  source 1..513: mol_type = other DNA
atgaatggagctataggaggtgaccttttgctcaattttcctgacatgtcggtcctagagcgccaaagggctcacctcaagtacctcaatcccacctttgattctcctctcgccggcttctttgccgattcttcaatgattaccggcggcgagatggacagctatctttcgactgccggtttgaatcttccgatgatgtacggtgagacgacggtggaaggtgattcaagactctcaatttcgccggaaacgacgcttgggactggaaatttcaaggcagcgaagtttgatacagagactaaggattgtaatgaggcggcgaagaagatgacgatgaacagagatgacctagtagaagaaggagaagaagagaagtcgaaaataacagagcaaaacaatgggagcacaaaaagcatcaagaagatgaaacacaaagccaagaaagaagagaacaatttctctaatgattcatctaaagtgacgaaggaattggagaaaacggattatattcat

SEQ ID NO: 44 - DNA, 27 bp, Synthetic construct
  source 1..27: mol_type = other DNA
tacccatacgatgtgccagattacgcc

SEQ ID NO: 45 - skipped

(19) United States
(12) Patent Application Publication
(10) Pub. No.: US 2025/0242060 A1
(43) Pub. Date: Jul. 31, 2025

(54) CHEMOGENETIC REGULATION OF PEPTIDE FUNCTION

(71) Applicant: The Regents of the University of Michigan, Ann Arbor, MI (US)

(72) Inventors: Wenjing Wang, Ann Arbor, MI (US); Peng Li, Ann Arbor, MI (US); Jiaqi Shen, Ann Arbor, MI (US); Lequn Geng, Ann Arbor, MI (US)

(21) Appl. No.: 18/855,956

(22) PCT Filed: Apr. 11, 2023
(86) PCT No.: PCT/US2023/018101

§ 371 Date: Oct. 10, 2024

Related U.S. Application Data
(60) Provisional application No. 63/329,736, filed on Apr. 11, 2022.

(51) Int. Cl. A61K 48/00; C12N 15/86

(52) U.S. Cl. CPC A61K 48/0066; A61K 48/0058; C12N 15/86; C12N 2750/14143; C12N 2830/002

(57) ABSTRACT

Provided herein are methods, compositions, kits and systems for regulation of peptide function. In particular, provided herein are methods, compositions, kits and systems comprising nucleic acid constructs and fusion proteins comprising chemically-activated protein domains (CAPs) that regulate the activity of a fused peptide in the presence of a ligand.

### CROSS-REFERENCE TO RELATED APPLICATIONS

The present application claims priority to U.S. Provisional Application Ser. No. 63/329,736 filed Apr. 11, 2022, the disclosure of which is herein incorporated by reference in its entirety.

### STATEMENT REGARDING FEDERALLY SPONSORED RESEARCH OR DEVELOPMENT

This invention was supported by Grant Nos. R01 AT011652 and R01 HL156989 awarded by the National Institutes of Health. The U.S. government has certain rights in the invention.

### FIELD

Provided herein are methods, compositions, kits and systems for regulation of peptide function. In particular, provided herein are methods, compositions, kits and systems comprising nucleic acid constructs and fusion proteins comprising chemically-activated protein domains (CAPs) that regulate the activity of a fused peptide in the presence of a ligand.

### BACKGROUND

Natural or engineered peptides serve biological functions by acting as partner in a dimerization pair¹, inhibitor of enzyme activity^{2,3}, and regulator of protein localization^{4,5}, degradation^{6,7}, and splicing⁸. Activation of peptide functionality with a user-defined signal, such as light or chemical, enables manipulation of diverse biological processes with temporal control. One way to achieve such control is by using chemically-modified synthetic peptides that incorporate removable protecting groups⁹ or an azobenzene-based chromophore¹⁰. While useful in vitro, these methods have limited use in endogenous biological environments due to the challenges of the delivery into cells and the subsequent degradation of external peptides. Another way to control peptide function is using genetically-encoded protein domains^{7,11}. By fusing a peptide to a protein that changes its conformation in the presence of light or small molecules, caging and uncaging of the peptide can be achieved. This approach allows genetic introduction of peptides into living organisms and cell type specific protein expression, and therefore is suited for biological studies. The second light, oxygen, voltage sensing domain from Avena sativa phototropin 1 (AsLOV2) has been used as a protein domain to control peptide functions^{2,6,11-16}. AsLOV2 works by controlling the accessibility of the N-terminus of a peptide that is fused to it. In the dark, the peptide is blocked by AsLOV2; in the light, a conformational change takes place in AsLOV2 and consequently, the peptide is released and becomes accessible^{17,18}. Although light provides fast temporal control, its use in non-transparent organisms is limited by poor tissue penetration¹⁹. In contrast, chemicals can be readily administered in animals, and many can penetrate cell membrane and deep tissue. A chemically-activated protein domain for controlling peptide function will complement its light-controlled counterpart and has useful applications for manipulating cellular processes. Chemical-dependent protein domains have been used to provide chemical control of protein proximity^{20,21} and conformation^{22,23}, but none have general applicability in controlling peptide functions. One example is a ligand-induced degradation (LID) system⁷, where a five-amino-acid peptide that can induce protein degradation is blocked by an FK506 binding protein (FKBP) until a small molecule, shield-1 (https://www.takarabio.com/products/inducible-systems/inducible-protein-stabilization/shield1), displaces the peptide from the ligand binding site. However, LID is unable to control other peptides and has limited potential for specific manipulation of cellular processes.

Accordingly, new protein-switches and gating modules based on small molecules are needed for controlling other peptides functions.

### SUMMARY

Provided herein are methods, compositions, kits and systems for regulation of peptide function. In particular, provided herein are methods, compositions, kits and systems comprising nucleic acid constructs and fusion proteins comprising chemically-activated protein domains (CAPs) that regulate the activity of a fused peptide in the presence of a ligand.

In some embodiments, the present invention provides a method of regulating the activity of a peptide, comprising generating a nucleic acid construct comprising a nucleic acid that encodes at least one chemically-activated protein domain (CAP) comprising a ligand binding site and a nucleic acid that encodes a peptide; administering the nucleic acid construct to one or more cells to generate a fusion protein wherein the fusion protein comprises the at least one CAP linked to the peptide; and administering a ligand to the one or more cells wherein the ligand binds to the ligand binding site of the CAP of the fusion protein, wherein the administering of the ligand increases activity of the peptide. In some embodiments, the at least one CAP comprises CapN that binds to the N-terminus of the peptide, and/or CapC that binds to the C-terminus of the peptide. In some embodiments, the administering the nucleic acid construct comprises direct injection of the nucleic acid construct, macromolecule-mediated liposomal and/or biopolymer gene delivery, plasmid delivery, and/or viral delivery. In some embodiments, the viral delivery is selected from the group consisting of adeno-associated viral (AAV) delivery, adenoviral delivery, lentivirus delivery, vaccina virus delivery and retroviral delivery. In some embodiments, the nucleic acid construct is stably expressed or transiently expressed. In some embodiments, expression is intracellular expression or extracellular expression.

In some embodiments, the ligand binding site comprises a FKBP binding domain. In some embodiments, the peptide is SsrA, a nuclear localization signal peptide (NLS), a nuclear export signal peptide, or TEV protease cleavage site. In some embodiments, the peptide is an enzyme activation peptide, an enzyme inhibition peptide, an enzyme regulation peptide, a binding peptide, a localization peptide, or a degradation peptide. In some embodiments, the ligand is shield-1 and/or aquashield-1.

In some embodiments, the administering is parenteral administering and/or non-parenteral administering. In some embodiments, the one or more cells are cells in vitro or cells in vivo. In some embodiments, the cells in vivo are neuronal cells or liver cells. In some embodiments, the nucleic acid construct comprises at least one nucleic acid selected from the group encoding Aga2p, a reporter gene, TetR DBD, SspB, Vp16, P2A, a linker, FLAG, UAS-mCherry and an internal ribosome entry site (IRES). In some embodiments, the nucleic acid construct further comprises a promoter nucleic acid selected from the group consisting of CMV, CAG and synapsin.

In some embodiments, methods of the present invention further a protein of interest (POI). In some embodiments, the POI is a transcription factor, a kinase, a gene editing enzyme, or a label. In some embodiments, the POI is evolved green fluorescent protein (EGFP). In some embodiments, methods of the present invention further comprise measuring the activity of the peptide wherein the measuring comprises measuring the localization, structure and/or function of the peptide. In some embodiments, the present invention provides a universal mechanism and/or module for temporal, user-controlled regulation of the structure, function and localization of an intracellular small peptide, large peptide or protein.

In some embodiments, the present invention provides a kit comprising: a nucleic acid construct, comprising: a nucleic acid that encodes at least one chemically-activated protein domain (CAP) comprising a ligand binding site; and a nucleic acid that encodes a peptide; a gene transfer reagent and/or vector: and a ligand that binds to the ligand binding site.

In some embodiments, the present invention provides a composition, comprising: a nucleic acid construct, comprising: a nucleic acid that encodes at least one chemically-activated protein domain (CAP) comprising a ligand binding site wherein said ligand is shield-1 and/or aquashield-1; a nucleic acid that encodes a peptide; and a gene transfer reagent and/or vector.

In some embodiments, the present invention provides a fusion protein, comprising a protein expressed by a nucleic acid that encodes at least one chemically-activated protein domain (CAP) comprising a ligand binding site wherein the ligand is shield-1, rapamycin and/or aquashield-1; and a peptide expressed by a nucleic acid wherein the peptide is regulated by the shield-1, rapamycin and/or aquashield-1.

### DESCRIPTION OF THE FIGURES

FIG. 1 shows a representative CapN system. a, Design of CAPs. CapN and CapC block the N- and C-terminus of a peptide, respectively. Addition of shield-1 releases the binding sequence from the ligand binding site, unblocking the peptide. b, Labeling and library selection for CapN-caged TEV protease cleavage site. CapN-tobacco etch virus protease cleavage site (CapN-TEVcs) is displayed on yeast surface by fusing to the yeast Aga2p protein. Accessibility of TEVcs is confirmed by protease cleavage. FLAG and HA signals indicate protein expression level and TEVcs cleavage, respectively. For library selection, a retained population is shown in rectangles on the FACS plots. (TEV protease cleavage site (ENLYFQ/G, cleaved between Q and G). FLAG and HA are epitope tags. c, Labeling scheme of CapN-caged SsrA. CapN-SsrA is displayed on yeast surface by fusing to the yeast Aga2p protein. Accessibility of SsrA is evaluated by binding to its binding partner, SspB. APEX2 labels protein within close proximity with biotin-phenol. FLAG and biotin signals indicate protein expression level and SsrA-SspB association, respectively. APEX2 is an engineered ascorbate peroxidase. FLAG is an epitope tag. d, CapN sequence before, during, and after directed evolution. Before directed evolution, the original linker and binding sequence (TRGVEEVAEGVVLL) (SEQ ID NO: 1) from LID was fused with TEVcs or SsrA to the C-terminus of FKBP. For directed evolution (libraries 1-4), the last six amino acids of the binding sequence was mutated to 6-9 random amino acids. The post-evolution sequence is the final CapN used for the rest of this study. Aga2p is the yeast protein for displaying CapN on the yeast surface. “X” indicates any of the twenty amino acids. Amino acids that are different from the original LID sequence are highlighted in red. e, FACS selection of CapN libraries to improve shield-1 dependence. Four libraries each with >10⁶ CapN variants were displayed on the yeast surface via fusion to the yeast Aga2p protein. Libraries were combined after the 1st round of selection. Values are percentage of cells in Q2 over (Q2+Q4). Sequencing results of post 4th round library are in FIG. 2. f, FACS analysis of CapN-caged TEVcs before and after directed evolution. Values are median HA intensity of FLAG-positive cells (Q2+Q4). Post-evolution CapN showed improved caging and dynamic range. This experiment was performed three times with similar results. Additional FACS plots are in FIG. 2. g, FACS analysis of CapN-caged SsrA before and after directed evolution, using labeling scheme as shown in c. Values are median biotin intensity of FLAG-positive cells (Q2+Q4). Post-evolution CapN showed both tighter caging and larger response to shield-1. This experiment was performed three times with similar results.

FIG. 2 shows ligand a binding site of FKBP and directed evolution results of CapN. related to FIG. 1. a, Crystal structure of FKBP12 (PDB:1FAP). The hydrophobic residues around the ligand binding site are shown in yellow and stick representation. b, Sequencing of forty clones from the post 4th round CapN library as shown in FIG. 1e. Twenty-three distinct sequences were identified. Clone #1 is the final CapN used for the rest of this study. c, FACS analysis of the most enriched eight clones, corresponding to clones #1-#8 shown in b. Values are median HA intensity of FLAG-positive cells (Q2+Q4). All eight clones showed similar results. Clone #1 is the final CapN used for the rest of this study. This experiment was performed once.

FIG. 3 shows shield-1 dose response characterization with CapN. a, FACS analysis of CapN-caged SsrA on yeast surface treated with different concentrations of shield-1. Three technical replicates were performed for each condition. Values are median biotin intensity of FLAG-positive cells (Q2+Q4). This experiment was performed twice with similar results. b, Dose-response curve using data from a. The median biotin signal is plotted against shield-1 concentration. Half maximum response was observed at 53 nM. Errors, s.e.m.

FIG. 4. Shows shield-1 reversibility characterization with CapN. a, Schematic of shield-1 reversibility characterization. Yeast cells were incubated with shield-1 for 10 min, followed by washing to remove excess shield-1. Yeast cells were then incubated at room temperature for 0-12 h before the accessibility of SsrA was evaluated using SspB-APEX2 and biotin-phenol labeling as shown in FIG. 1c. b, FACS analysis of the yeast cells from a. Values are median biotin intensity of FLAG-positive cells (Q2+Q4). No decrease in biotin signal was seen even after cells were incubated for 12 h without shield-1. This experiment was performed once.

FIG. 5 shows engineering of the CapC system. a, CapC sequence before, during, and after directed evolution. Before directed evolution, a flexible linker (SGAGSGGSGTGSGSGGS) (SEQ ID NO: 2) and the last six amino acids (RYSPNL) (SEQ ID NO: 3) from the post-evolution CapN binding sequence was fused with SsrA to the N-terminus of FKBP. For directed evolution (libraries 1-3), amino acids highlighted in red were mutated randomly into any of the twenty amino acids. The post-evolution sequence is the final CapC used for the rest of this study. Aga2p is the yeast protein for displaying CapC on the yeast surface. “X” indicates any of the twenty amino acids. Amino acids that are different from the post-evolution CapN sequence are highlighted in red. b, Labeling and library selection scheme for CapC-caged SsrA. SsrA-CapC is displayed on yeast surface and accessibility of SsrA is evaluated using the same scheme as shown in FIG. 1c. For library selection, retained population is shown in rectangles on the FACS plots. c, FACS selection of CapC libraries to improve shield-1 dependence. Pre- and post-evolution CapC are also shown for comparison. Three libraries each with >10⁶ CapN variants were displayed on the yeast surface via fusion to the yeast Aga2p protein. Libraries were combined for selection. Values shown in plots represent the percentage of cells in Q2 over (Q2 Q4). Sequencing results of post 2nd round library and additional FACS plots are in FIG. 6. This experiment has been performed once.

FIG. 6 shows all-atom molecular dynamics simulations. Results from a 2 microsecond molecular dynamics simulations of FKBP and a capped ArgTyrSerProAsnLeu (SEQ ID NO: 4) peptide in 150 mM buffer. a, the central configurations for the top 5 clusters (Rank 1-5) obtained from RMSD clustering indicate direct interactions between Leu6 of the peptide (shown in a “licorice” representation; cap residues are shown in green, other atoms in CPK colors with gray carbons) and the F36V binding site of FKBP (shown as van-der-Waals spheres). The secondary structure of the FKBP protein is shown in a cartoon representation with red α-helices and yellow β-sheets. b, RMSD time traces with respect to the structures shown in a indicate the longevity of the respective conformations within the simulations. RMSD's of 0 indicate the simulation time points corresponding to the structures in a. A horizontal dashed line indicates the 1.5 Å cutoff used for clustering. c, time traces of the center of mass distances between each individual sidechain of the peptide and the sidechain of the F36V binding site indicate a persistent proximity of Leu6 to the binding site for a large fraction of the simulation trajectory (distances of 5-6 Å). Fractions of the simulation trajectory with close proximity of Leu6 to the F36V binding site include all configurations associated with the top 5 clusters shown in a.

FIG. 7 shows directed evolution results of CapC. Related to FIG. 5. a, Sequencing of twenty clones from the post 2nd round CapC library as shown in FIG. 5c. Eighteen distinct sequences were identified and characterized. One sequence with early stop codon is not shown. Clone #18 is the final CapC used for the rest of this study. b, FACS analysis of the eighteen clones shown in a. Values are median HA intensity of FLAG-positive (Q2+Q4) cells. Clone #18 is the final CapC used for the rest of this study. This experiment was performed once.

FIG. 8 shows a comparison of single CapN, single CapC, and tandem CAPs in caging TEVcs. a, Schematics of the three constructs tested. CapN-TEVcs-CapC is the combined use of both post-evolution CAPs. Aga2p is the yeast protein for displaying constructs on the yeast surface. TEVes, TEV protease cleavage site (ENLYFQ/G, cleaved between Q and G). FLAG and HA are epitope tags. b, FACS plots of the three constructs shown in a. Values are median biotin intensity of cells in Q2 and Q4. This experiment used a stronger TEV protease condition than FIGS. 1e and 1f See method section for details.

FIG. 9 shows applying CAPs for shield-i-induced protein translocation. a, Schematics of shield-1 induced protein translocation to plasma membrane. Protein of interest (EGFP as example) fused to SspB is translocated to the plasma membrane when SsrA is uncaged from CAPs. mCherry is used as a membrane protein marker. Transmembrane domain is CAAX. POI, protein of interest. b, Representative fluorescence microscopy images of HEK293T cells expressing the constructs shown in a. Addition of shield-1 induces EGFP translocation to the plasma membrane within seconds. The right panel shows the intensity profiles of mCherry and EGFP along the red line in images. Scale bar, 20 μm. This experiment was performed once. c, Schematics of shield-1 induced protein translocation from plasma membrane. Protease cleavage of TEVcs allows POI (EGFP as example) to be removed from the plasma membrane. mCherry is used as a protease expression marker. Transmembrane domain is CAAX. TEVcs, TEV protease cleavage site (ENLYFQ/M, (SEQ ID NO: 5) cleaved between Q and M). POI, protein of interest. d, Representative fluorescence microscopy images of HEK 293T cells expressing the constructs shown in c. Addition of shield-1 depletes EGFP from the plasma membrane. Scale bar, 20 μm. Additional images are in FIG. 8. This experiment was performed twice with similar results. e, Schematic of CAPs controlling nuclear localization signal peptide. Shield-i-dependent uncaging of NLS brings POI (EGFP as example) from cytosol to nucleus. mCherry is used to indicate the cytosol. NLS, nuclear localization signal peptide (PKKKRKV) (SEQ ID NO: 6). POI, protein of interest. NES, nuclear export signal peptide (LQLPPLERLTLD) (SEQ ID NO: 7). PKIt NES, truncated cAMP-dependent protein kinase inhibitor alpha (PKIt) NES (LALKLAGLDI) (SEQ ID NO: 8). f, Left: Representative fluorescence microscopy image of HEK 293T cells expressing the constructs shown in e. Scale bar, 20 m. Additional images are in FIG. 10. This experiment was performed three times with similar results. Right: Quantification of EGFP total intensity distribution. The ratio is calculated by the EGFP total intensity in cytosol to that in whole cell. The cytosol and whole cell is determined by mCherry. The center lines indicate mean values of the ratio. P value is determined by unpaired two-tailed t-tests. ****P<0.0001.

FIG. 10 shows additional images of FIG. 9. Left: Four additional views for each condition. Right: Negative control with CAPs caging SsrA instead of TEVcs; Shield-1 addition did not result in increased cytosolic pattern of EGFP. This further supports that the translocation from membrane is due to TEV protease cleavage. Scale bar, 20 μm.

FIG. 11 shows additional fluorescence microscopy images of HEK 293T cells expressing the constructs shown in FIG. 9e under+/−shield-1 conditions. Scale bar, 20 μm.

FIG. 12 shows application of CAPs to shield-1 induced transgene transcription. a, Schematics of shield-i-induced gene transcription. Uncaging of SsrA reconstitutes the split transcription factor and results in reporter gene (mCherry as example) expression. EGFP is used as an expression marker for SspB and transcription-activation domain. Transcription-activation domain is VP16 for all following experiments. DNA-binding domain (DBD) is specified under each experiment. b, Summary of main constructs tested. Amino acid sequences of SsrA are highlighted. DBD, DNA-binding domain. P2A, a self-cleaving peptide. IRES, internal ribosome entry site. c, Quantification of mCherry expression level for constructs shown in b. Numbers on the plot are the ratio of mean mCherry intensity of + shield-1 to that of − shield-1 conditions for each construct. The center lines indicate mean values of mCherry intensity. Images are shown in FIGS. 11, 13 and 17. For this experiment, Gal4 was used as DBD, and UAS-mCherry was used as reporter gene. n=12 for all conditions. This experiment was performed three times with similar results. d, Representative fluorescence microscopy image of HEK 293T cells expressing the best-performing non-single-component construct, u3. Same DBD and reporter gene as in c. Scale bar, 20 μm. FLAG is an epitope tag. e, Representative fluorescence microscopy images of rat cortical neurons expressing the single-component construct, u4, and quantification of mCherry expression level. For this experiment, TetR was used as DBD, and TRE-mCherry was used as reporter gene. The number on the plot is the ratio of mean mCherry intensity of + shield-1 to that of − shield-1 conditions. The center lines indicate mean values of mCherry intensity. n=5 for both conditions. Scale bar, 100 m. Additional images used for quantification are in FIG. 18. This experiment was performed twice with similar results. P values are determined by unpaired two-tailed t-tests. **P<0.01; ***P<0.001; ****P<0.0001.

FIG. 13 shows images of constructs u1 and u2 in FIGS. 12b and 12c. Fluorescence microscopy images of HEK 293T cells expressing the constructs shown and UAS-mCherry reporter gene. These images were used for quantification of constructs u1 and u2 in FIG. 12c. All scale bars, 50 μM.

FIG. 14 shows images of construct u3 in FIGS. 12b and 12c, and two other constructs with different SsrA sequences. Fluorescence microscopy images of HEK 293T cells expressing the constructs shown and UAS-mCherry reporter gene. These images were used for quantification of construct u3 in FIG. 12c. Two other truncations shown here (truncating one or both N-terminal alanine from SsrA) did not show improvement. All scale bars, 50 μM.

FIG. 15 shows the effect of shield-1 on transcription-activation domain expression. No difference was found between the + shield-1 and − shield-1 conditions. All scale bars, 5 μM.

FIG. 16 shows gene expression at different shield-1 concentrations with construct u3 in FIG. 12a, Quantification of mCherry expression level under different shield-1 concentrations. 50 μM or above induced robust gene expression. P values are determined by unpaired two-tailed t-tests. ***P<0.001; NS, not significant. n=12 for all conditions. b, Fluorescence microscopy images of HEK 293T cells used for quantification in a, showing mCherry (reporter gene expression) only. Gal4 was used as DBD, and UAS-mCherry was used as reporter gene. Scale bar, 50 μm. c, Fluorescence microscopy images of HEK 293T cells in the same experiment in b, showing EGFP (transcription-activation domain expression) only. Note that the difference in EGFP signal does not indicate an actual difference in gene expression. Scale bars, 50 μM.

FIG. 17 shows gene expression after incubating with shield-1 for different amounts of time using construct u3 in FIG. 12b. a, Schematics of the experiment. HEK 293T cells expressing construct u3 were incubated with 100 nM or 1000 nM shield-1 for different amount of time. Cells were then washed and incubated without shield-i, and imaged 24 h after initial shield-1 addition b, Quantification of mCherry expression level with different shield-1 incubation time, at 100 nM or 1000 nM shield-1 concentrations. P values are determined by unpaired two-tailed t-tests. *P<0.05; **P<0.01; ***P<0.001; ****P<0.0001; NS, not significant. n=12 for all conditions. c, Representative fluorescence microscopy images of HEK 293T cells used for quantification in b. For this experiment, Gal4 was used as DBD, and USA-mCherry was used as reporter gene. Scale bar, 50 m.

FIG. 18 shows images of constructs u4 and u5 in FIGS. 12b and 4c. Fluorescence microscopy images of HEK 293T cells expressing the constructs shown and UAS-mCherry reporter gene. These images were used for quantification of constructs u4 and u5 in FIG. 12c. All scale bars, 50 μm.

FIG. 19 shows additional images of FIG. 12e. Four additional views for each shield-1 condition are shown. Scale bar, 100 μm.

FIG. 20 shows aqua-shield-1 (https.//cheminpharma.com/product/as1-50-mm-2/). induced transgene expression in mice tissues. a, Timeline for the aquashield-1-induced transgene expression in mouse brain. Aquashield-1 is locally administered to mice (1 μL, 1 mM). b, Representative fluorescence microscopy images of brain sections of the lateral hypothalamic area and quantification of total number of cells expressing mCherry. Numbers on the plot are the ratio of mean cell count of + aquashield-1 to that of − aquashield-1 conditions. The center lines indicate mean values of cell count. n=3 for both conditions. Scale bar, 200 μm. c, Timeline for the aquashield-1-induced transgene expression in mouse liver. Aquashield-1 is administered to mice via two intraperitoneal (IP) injections (40 mg/kg) with 24 hours apart. d, Representative fluorescence microscopy images of liver sections from injection site and quantification of total number of cells expressing mCherry. Numbers on the plot are the ratio of mean cell count of + aquashield-1 to that of − aquashield-1 conditions. The center lines indicate mean values of cell count. n=3 for both conditions. Scale bar, 200 μm. P values are determined by unpaired two-tailed t-tests. *P<0.1; ***P<0.001.

FIG. 21 shows a representative gating strategy for single cell analysis.

FIG. 22 shows a schematic diagram of a split NanoLuc assay for detection of CapC-caged met-enkephalin activation in a 2-chain design (left panel) and a 1-chain design (right panel).

FIG. 23 shows quantification of the split NanoLuc assay for the 1-chain design provided in FIG. 22, right panel. The X-axis provides different experimental conditions. The Y-axis provides luminescence signal in relative luminescence units (RLU).

FIG. 24 shows activity of CapC-caged met-enkephalin detected by the GloSensor cAMP assay. RLU, relative luminescence unit.

### DEFINITIONS

To facilitate an understanding of the present disclosure, a number of terms and phrases are defined below. While the invention will be described in conjunction with certain representative embodiments, it will be understood that the invention is not limited to these illustrative examples. One skilled in the art will recognize many methods and materials similar or equivalent to those described herein may be used in the practice of the present invention. The present invention is in no way limited to the methods and materials described.

Unless defined otherwise, technical and scientific terms used herein have the meaning commonly understood by one of ordinary skill in the art to which this invention belongs. Although any methods, devices, and materials similar or equivalent to those described herein can be used in the practice of the invention, certain methods, devices, and materials are described herein. It is further to be understood that all base sizes or amino acid sizes, and all molecular weight or molecular mass values, given for nucleic acids or polypeptides are approximate, and are provided for description.

As used in this disclosure, including the appended claims, the singular forms “a,” “an,” and “the” include plural references, unless the content clearly dictates otherwise, and are used interchangeably with “at least one” and “one or more.”

As used herein, the term “about” represents an insignificant modification or variation of the numerical value such that the basic function of the item to which the numerical value relates is unchanged.

As used herein, “protein” is used synonymously with “peptide,” “polypeptide,” or “peptide fragment.” A “purified” polypeptide, protein, peptide, or peptide fragment is substantially free of cellular material or other contaminating proteins from the cell, tissue, or cell-free source from which the amino acid sequence is obtained, or substantially free from chemical precursors or other chemicals when chemically synthesized.

As used herein, “modulate” means to alter, either by increasing or decreasing, the activity of a gene or protein. The term “inhibit”, as used herein, means to prevent or reduce the activity of gene or protein.

As used herein, the term “bioactivity” indicates an effect on one or more cellular or extracellular process (e.g., via binding, signaling, etc.) that can impact physiological or pathophysiological processes.

Ranges provided herein are understood to be shorthand for all of the values within the range. For example, a range of 1 to 50 is understood to include any number, combination of numbers, or sub-range from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise). Any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “in vitro” refers to an artificial environment and to processes or reactions that occur within an artificial environment. In vitro environments can consist of, but are not limited to, test tubes and cell lysate. The term “in vivo” refers to the natural environment (e.g., an animal or a cell) and to processes or reaction that occur within a natural environment.

### DETAILED DESCRIPTION OF THE DISCLOSURE

Provided herein are methods, compositions, kits and systems for regulation of peptide function. In particular, provided herein are methods, compositions, kits and systems comprising nucleic acid constructs and fusion proteins comprising chemically-activated protein domains (CAPs) that regulate the activity of a fused peptide in the presence of a ligand.

Natural or engineered peptides serve important biological functions. A general approach to achieve chemical-dependent activation of short and long peptides provides for spatial and temporal control of cellular processes. In some embodiments, the present invention provides a pair of chemically-activated protein domains (CAPs) for controlling the accessibility of both the N- and C-termini of a peptide. In experiments conducted in the development of embodiments of the present invention, CAPs were identified through directed evolution of an FK506 binding protein (FKBP). By fusing peptides to one or both CAPs, their function is blocked until a small molecule displaces them from the FKBP ligand binding site. The present invention provides CAPs that are applicable to a range of short and long peptides, including a protease cleavage site, the SsrA (10S RNA, TmRNA) peptide that dimerizes with the SspB protein, and a nuclear localization signal (NLS) peptide, with a shield-1 dependent dynamic range up to 156-fold. In some embodiments, CAPs systems, methods, compositions and kit find use in mammalian cell culture, and in diverse tissues in living animals, thereby providing chemogenetic approach to control peptide activity in vitro and in vivo.

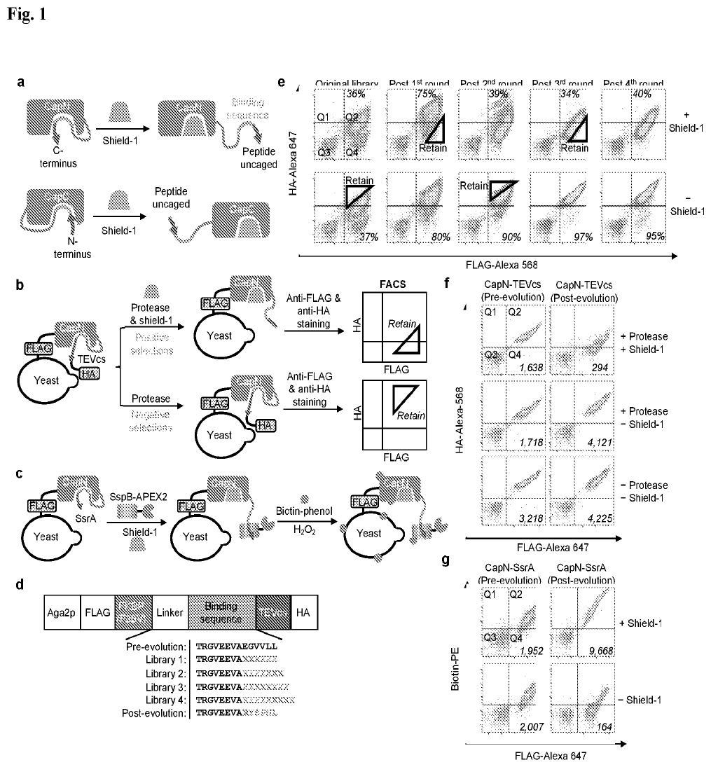

In experiments conducted in development of the present invention, CAPs were generated via directed evolution and include two protein domains, CapN and CapC, for blocking the N- and C-terminus of a peptide, respectively (FIG. 1a). Addition of shield-1 relieves steric blocking and activates peptide functions. In some embodiments, the present invention provides CAPs to cage the tobacco etch virus protease cleavage site (TEVcs), the SsrA (tmRNA, or 10Sa P1TA) peptide, and a nuclear localization signal peptide (NLS). In some embodiments, CAPs find use in mammalian cell culture, for example, HEK cells and neuronal cells. In some embodiments CAPs find use in tissue of living animals, for example in brain and liver tissue, thereby comprising both in vitro and in vivo utilities to control peptide activity. CAPs provide broad applicability in caging short and long peptides, including TEVcs, SsrA, and NLS. In some embodiments, CAPs are used to translocate proteins to diverse cellular locations and to control gene transcription in a chemical-dependent manner in a variety of biological contexts.

CAPs provide multiple advantages over existing technology. Compared with existing genetic methods of controlling peptide functions, which are largely light-based^{2,6,11-15,27}, CAPs provide an alternative approach by using a small molecule, shield-1 or aquashield-1. Shield-i has cell permeability and can be administered systematically in living animals. This enables the use of CAPs in most tissues of a living organism, including those that are difficult or otherwise too invasive to reach by light, such as the brain and the liver. Even though other chemical-dependent protein domains have been described, CAPs are to provide general applicability for different peptides, for example, to cage TEVcs and SsrA, regulate NLS, and control gene transcription. In some embodiments, tandem CAPs provide improved caging efficiency compared to single CAP.

For example, SsrA caged by CAPs in tandem provides up to 156-fold chemical-dependence. The caging strategy using two protein domains provides multiple advantages. For example, undesired leakage is a long-standing challenge to use of protein cages; a tandem caging strategy provides a solution to this problem for protein engineers.

In some embodiments, the caging mechanism of CAPs is transferrable to other peptides, with fine-tuning of the peptide or the binding sequence to acquire high signal-to-noise ratio, similar to the use of AsLOV2,^{13,16,32} for example with essential residues of a functional peptide located immediate to the CAPs domain. CAPs provide a different approach to temporally control peptide functions, with the capacity to cage larger polypeptides or proteins with effector sites on are located near the termini of the sequence in parallel to use of AsLOV2 as the cage^{29,33-35}. The CPs engineering strategy is also applicable to other chemical-dependent protein domains such as the hepatitis C virus protease NS3a^{36,37} and the BCL-xL protein³⁸. Engineering of these domains as general protein cages provides multiplexed control of cellular processes and expand the availability of chemogenetic technology.

### Methods

In some embodiments, the methods of the present invention find use in research when, for example, temporal control is desired to regulate peptide and/or protein localization, structure and function by the administration of a small molecule. For example, in some embodiments, CapC is used to regulate opioid peptide display on the cell outer membrane thereby enabling characterization of endogenous Gi-coupled opioid receptor activity. In some embodiments, the methods of the present invention find use in therapy when, for example, temporal control is desired to regulate peptide and/or protein localization, structure and function by the administration of a small molecule to activate a targeted pathway.

The present disclosure contemplates the use of any genetic manipulation for use in modulating the expression of a peptide of interest. Delivery of nucleic acid construct to cells in vitro or in vivo may be conducted using any suitable method. A suitable method is one that introduces the nucleic acid construct into the cell such that the desired event occurs (e.g., expression of a CAP-peptide construct). Introduction of molecules carrying genetic information into cells is achieved by any of various methods including, but not limited to, directed injection of naked DNA constructs, and macromolecule mediated gene transfer using, for example, liposomes, biopolymers, and the like. In some embodiments, methods of the present invention provide gene delivery vehicles derived from viruses, including, but not limited to, adenoviruses, retroviruses, lentivirus viruses, vaccinia viruses, and adeno-associated viruses. Vectors may be administered to a subject in a variety of ways. For example, in some embodiments of the present disclosure, vectors are administered into tissue associated by direct injection. In other embodiments, administration is via the blood or lymphatic circulation.

The present disclosure further provides pharmaceutical compositions e.g., comprising the chemogenic regulation compounds and their described above. The pharmaceutical compositions of the present disclosure may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intra-arterial, subcutaneous, intraperitoneal or intramuscular injection or infusion; or intracranial, e.g., intrathecal or intraventricular, administration. In certain embodiments, the nucleic acid constructs and/or their products are administered by methods that bypass the blood brain barrier (BBB) including, for example, direct application to the surface of the CNS, to the parenchyma of the CNS, to the ventricles of the CNS, and to the cerebrospinal fluid (CSF) of the CNS. In particular, intrathecal and epidural administration may be achieved by single shot, a series of single shots, and/or by continuous administration to the CSF. In certain embodiments, continuous administration to the CSF is provided by a programmable external pump. In other embodiments, continuous administration is provided by a programmable implantable pump. Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients. Agents that enhance uptake of nucleic acid constructs at the cellular level may also be added to the pharmaceutical and other compositions of the present disclosure. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

### Kits

The present disclosure provides kits comprising components of the chemogenetic peptide activation assays described herein. Such kits may comprise, for example, two or more of a nucleic acid that encodes at least one chemically activated protein domain (CAP) comprising a ligand binding site, a nucleic acid that encodes a peptide, a ligand that binds to the ligand binding site, a gene transfer vector and/or reagent, a stabilizer, a buffer, instructions, positive and negative control reagents, and ligands to identify possible sources of error and contamination. In some embodiments, a positive control peptide achieves statistical significance indicating that the positive control experiment performs to specification. In some embodiments, a kit of the present invention comprises calibration reagents and ligands including, for example, a luminescence calibration reagent. In some embodiments, a kit comprises customizable options. In some embodiments, a kit of the present invention comprises frozen (e.g., prepared with glycerol for storing at −20 C degree) components and reagents, and/or desiccated reagents and components.

### Compositions and Reaction Mixtures

The present disclosure provides compositions and reaction mixtures comprising components of the assays described herein. Such compositions and reaction mixtures may comprise, for example, two or more of a nucleic acid that encodes at least one chemically activated protein domain (CAP) comprising a ligand binding site, a nucleic acid that encodes a peptide, a ligand that binds to the ligand binding site, a gene transfer vector and/or a reagent, a stabilizer, a buffer, instructions, positive and negative control reagents, and ligands to identify possible sources of error and contamination. In some embodiments, compositions and reaction mixtures of the present invention comprise a fusion protein product of one or more of nucleic acid constructs provided herein.

### EXPERIMENTAL EXAMPLES

The following examples are provided to demonstrate and further illustrate certain embodiments and aspects of the present disclosure and are not to be construed as limiting the scope thereof.

### Example 1—Design and Directed Evolution of CapN

FIG. 1a shows a CAP for caging the N-terminus of a peptide. The design of CapN is an improvement on LID⁷, that is based on a mutant of an FK506 binding protein, FKBP12(F36V) referred to as FKBP. In LID, a 5-amino-acid peptide degradation sequence is blocked by FKBP via fusion to a binding sequence that interacts with FKBP. The degradation sequence is activated when an FKBP ligand, shield-1, is added to displace the binding sequence. The ligand for LID, shield-1, has sub-nanomolar affinity for FKBP, which is over 1000-fold higher than that for the wild-type FKBP²⁴. Therefore, shield-1 has minimal interference with the physiological functions of endogenous FKBP. Shield-1 is membrane permeant and has been previously used in live animals²⁵.

The ability of LID to cage two short peptides that have been previously shown to be controllable by the AsLOV2 domain was tested. A heptapeptide, TEVcs²⁶, and an octapeptide, modified SsrA²⁷, were displayed on yeast surface, and the accessibility was evaluated using a protease cleavage assay (FIG. 1b) and a binding assay (FIG. 1c), respectively. The degradation sequence (RRRGN) in LID was replaced with TEVcs or SsrA, and the remainder of the binding sequence and linker was retained (FIG. 1d). A shield-1 dependence for either TEVcs or SsrA was not observed indicating that LID is ineffective in introducing steric blocking to peptides other than those of original degradation sequence.

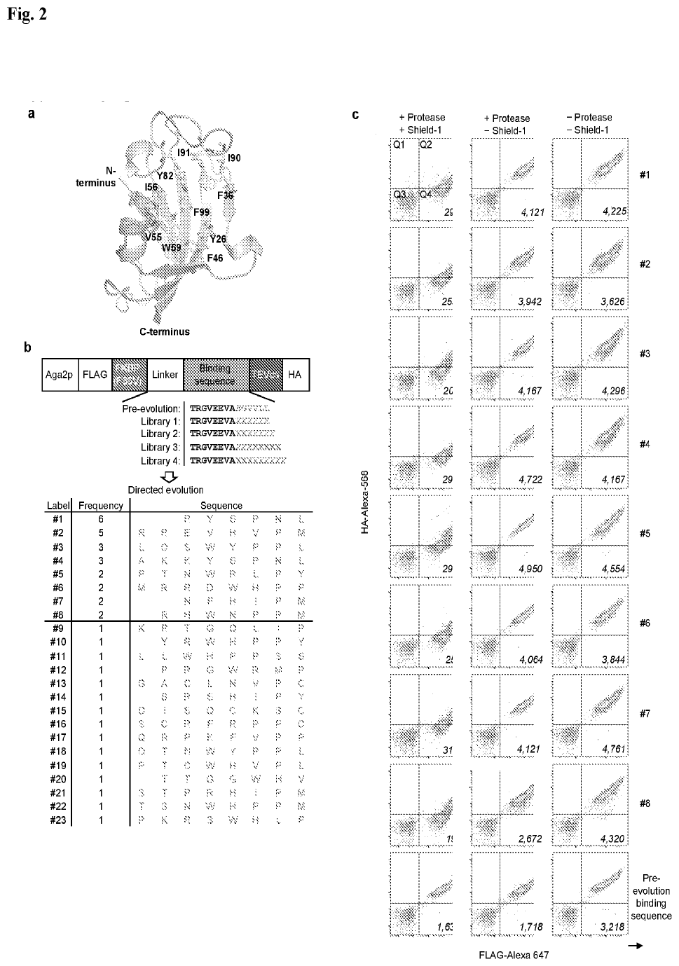

To test that the amino acids near the C-terminus of the binding sequence are critical for interacting with the hydrophobic ligand binding site of FKBP (FIG. 2), these residues were tuned. Yeast surface-based directed evolution was applied to improve the shield-1 dependence of CapN, using TEVcs as the caged peptide. A first library was generated by introducing site-saturated mutagenesis to the last 6 amino acids of the binding sequence immediately before TEVcs (FIG. 1d). To ensure sufficient length for binding and caging, 3 more libraries were constructed by adding 1, 2, or 3 additional amino acids to the randomized binding sequence (FIG. 1d). Four libraries were selected according to the scheme shown in FIGS. 1b and 1e. One round of negative selection was performed to retain CapN variants with tight caging in the absence of shield-1 (high expression and low cleavage). This also ensured all clones entering the second round of selection expressed the DNA construct in its entirety with no early stop codons. A round of positive selection was then performed to enrich clones that efficiently uncage TEVcs upon addition of shield-1. After 2 rounds of selection, the libraries of CapN variants showed shield-1 dependence. Two additional rounds of selection were performed to further enrich the targeted clones.

After 4 rounds of directed evolution, sequencing of 40 individual clones revealed 23 distinct sequences enriched in hydrophobic amino acids (FIG. 2). Eight clones were characterized that appeared more than once and with similar level of improvement. The most enriched clone was as the post-evolution CapN (FIG. 1d). Compared with the pre-evolution CapN, the post-evolution protein showed both tighter caging and larger response to shield-1 (FIG. 1f). When the same post-evolution CapN was applied to SsrA, enhanced caging and larger dynamic range were also observed (FIG. 1g). These results demonstrate the general applicability of CapN in controlling short peptides.

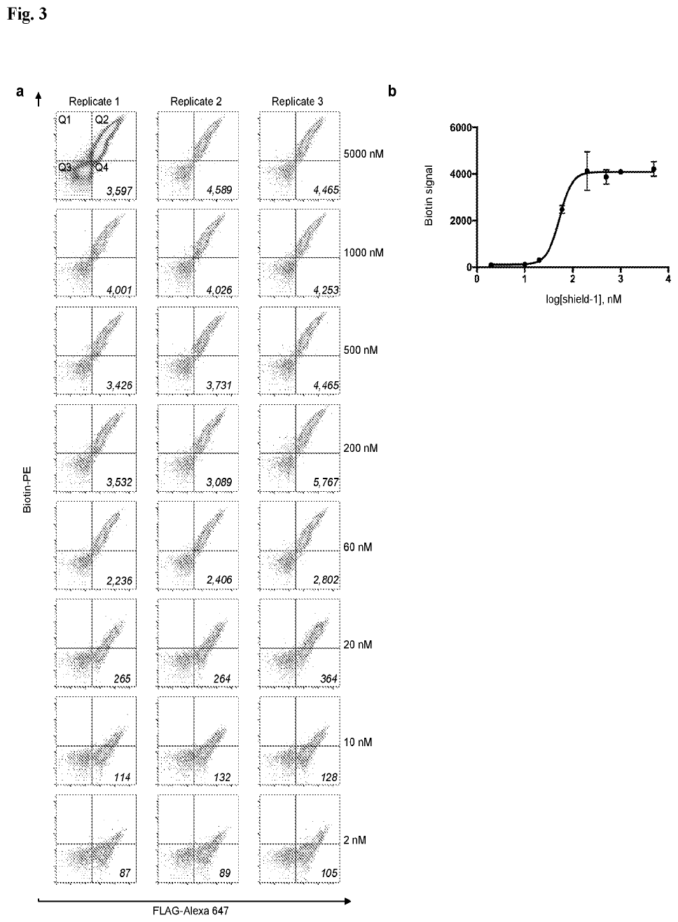

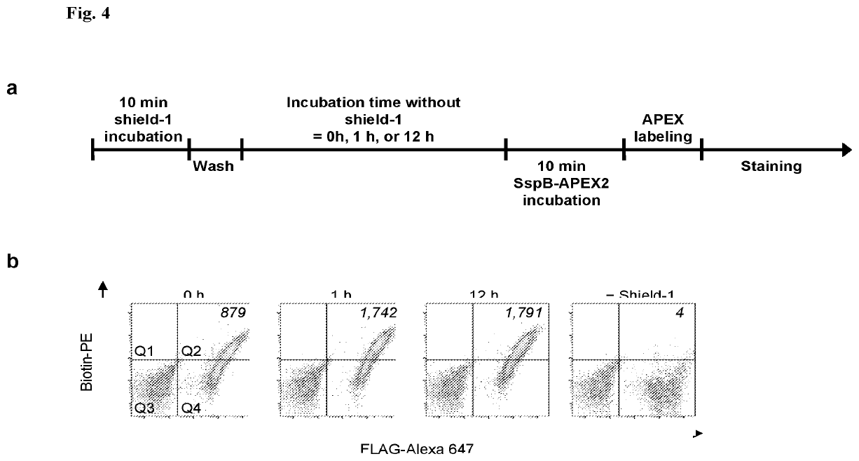

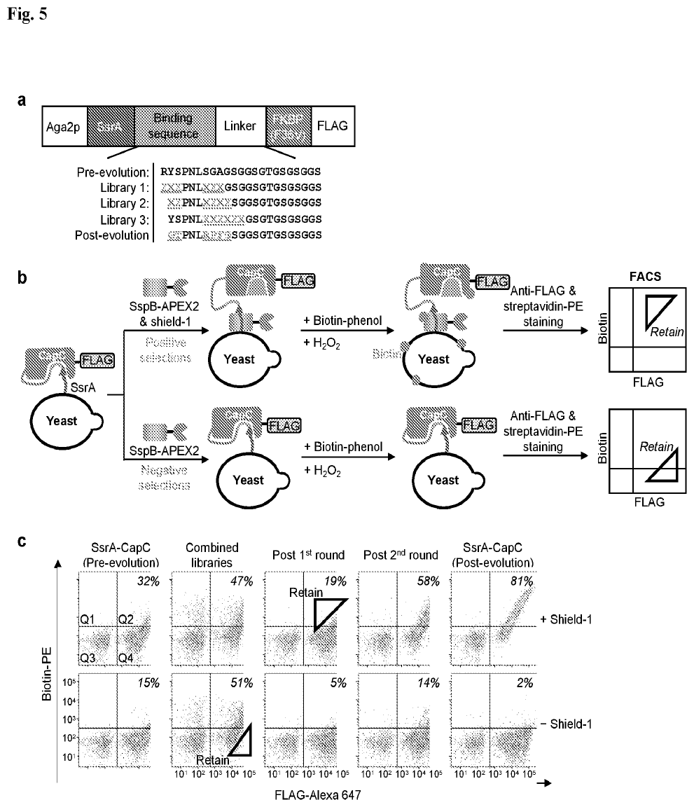

The concentration of shield-1 needed to activate CapN was then characterized (FIG. 3). Using the same yeast surface binding assay, the dose-response curve showed half maximum response at 53 nM, demonstrating high affinity of shield-1 towards CapN. Whether the binding between shield-1 and CapN is reversible was then assessed. Even 12 h after washing away shield-1, CapN remained open (FIG. 4), indicating that shield-1 stably binds to CapN, providing a CapN an irreversible “on” switch.

### Example 2—Design and Directed Evolution of CapC

After the development of CapN, an analogous CAP that cages the C-terminus of a peptide (CapC) was designed. Cagiong of a peptide is often most effective when the key residues on a peptide are closely bound to the CAP proteins. Peptides with a crucial C-terminal sequence may be better caged by CapC rather than CapN. As well, CapN and CapC may be used in tandem to further increase the caging efficiency, and to reduce “leaking” in the absence of shield-1. These features are critical for applications in live animals where the protein may be expressed for weeks to months before controlled protein activation is desired.

To test whether the shield-1 binding sequence functions regardless of its connectivity to FKBP, it was fused to the N-terminus of FKBP with a flexible linker (FIG. 5a) and its efficacy treated in caging the SsrA peptide using the same binding assay for testing CapN (FIG. 5b). SsrA was selected because CapN shows a larger dynamic range (difference between “+ shield-1” and “− shield-1” conditions) in caging SsrA than caging TEVcs, thereby facilitating selection during directed evolution. To design the linker, the distance from the N-terminus of FKBP to its ligand binding site was estimate, and a flexible glycine- and serine-rich linker used to span the distance. An initial CapC construct showed shield-1 dependence (FIG. 5c, first column) with a dynamic range was smaller than CapN.

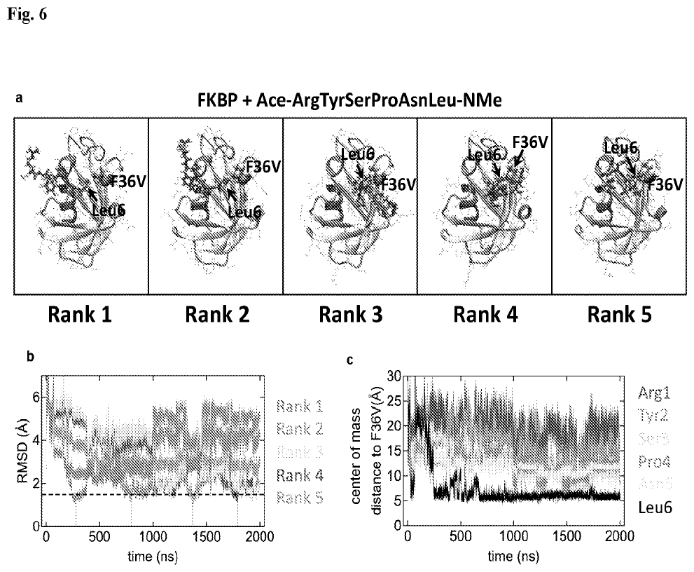

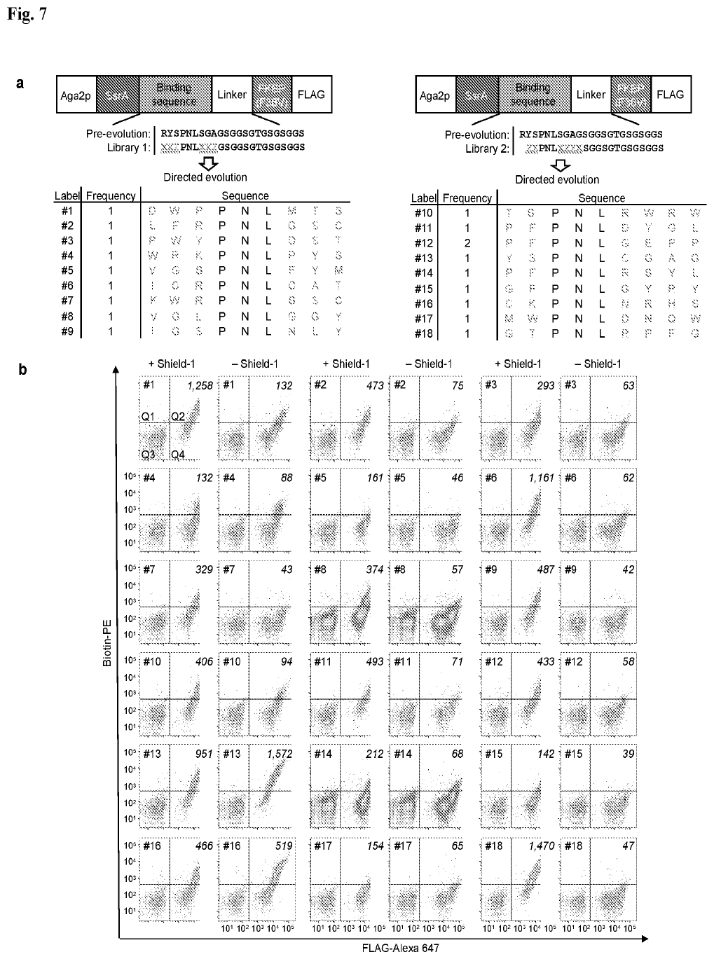

Directed evolution was used to enhance the dynamic range of CapC. To generate libraries, site-saturated mutagenesis was applied to two critical regions simultaneously (FIG. 5a): (1) the first 2 or 3 amino acids of the binding sequence; the region adjacent to the peptide target is the most critical in pulling it deeper into the FKBP ligand binding site; (2) the first several amino acids on the linker just after the binding sequence; this region may be critical in positioning the binding sequence towards the FKBP ligand binding site. The last 3 amino acids (Pro-Asn-Leu) of the binding sequence were retained because they provide a hydrophobic surface which important for binding. The importance of the hydrophobic leucine residue was supported by an all-atom molecular dynamics simulation that showed leucine has a high tendency to interact with the FKBP hydrophobic ligand binding site (FIG. 6). Three libraries were combined and selected according to the scheme shown in FIGS. 5b and 5c. One round of negative selection to retain CapC variants was performed with tight caging in the absence of shield-1 (high expression and low biotin signal). Then, one round of positive selection was performed to retain variants that uncage the peptide in the presence of shield-1 (low expression and high biotin signal). After 2 rounds of sorting, shield-1 dependence of the CapC libraries was enhanced. We characterized 20 individual clones and identified one with tight caging and large dynamic range as a post-evolution CapC (FIG. 5a, 5c, and FIG. 7).

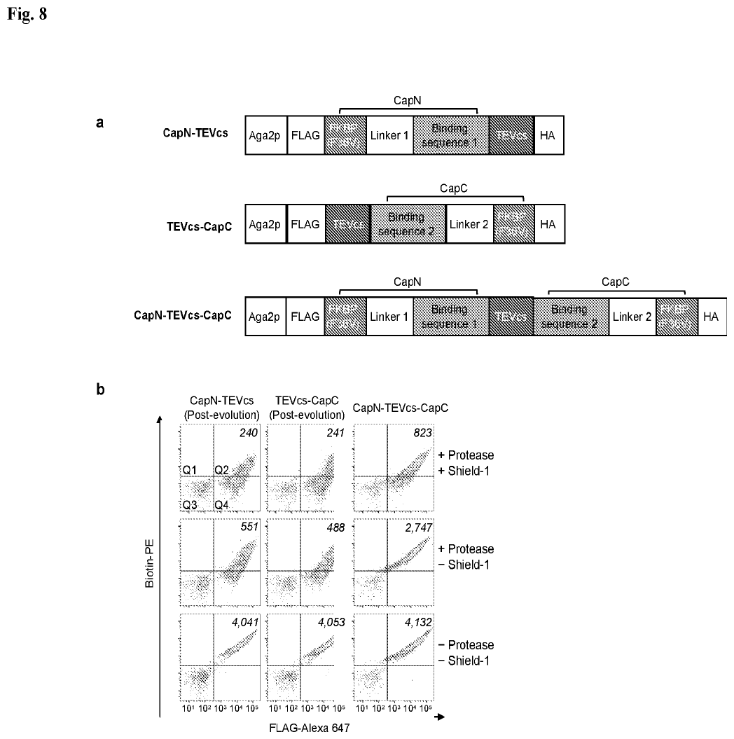

Because unwanted leakage is a common issue for protein cages, CapN and CapC used together were tested for tighter caging and reduce background. Using TEVes as the caged peptide, the tandem use of both CAPs was found to significantly reduce the leakage compared to using either CapN or CapC alone, while still able to be uncaged by shield-1 (FIG. 8), indicating that using both CAPs simultaneously enhances caging efficiency and reduces unwanted leaking. Accordingly, tandem CAPs were used for the following experiments in cell culture and in animals.

### Example 3—CAPs Control Protein Translocation to Plasma Membrane in HEK 293T Cells

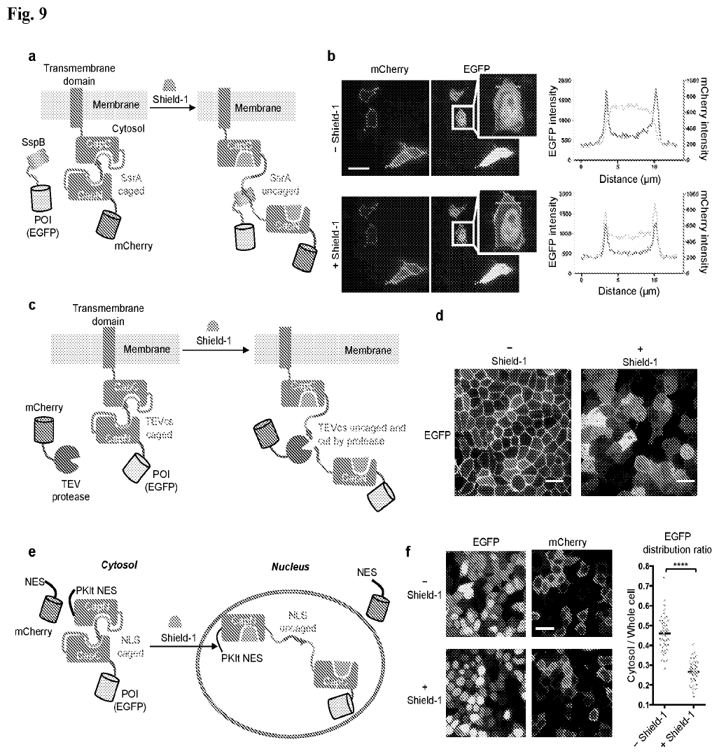

Protein transportation to different subcellular compartments is closely linked to their function, and controlled localization to the plasma membrane has been used to manipulate cellular processes such as phagocytosis²⁸ and calcium influx²⁹. To test use of CAPs for protein translocation, a shield-1 induced membrane localization system was designed (FIG. 9). In this design, CAPs-caged SsrA peptide is localized on the plasma membrane of HEK 293T cells by fusing to a transmembrane domain. The interacting partner of SsrA, SspB protein, is fused to the protein of interest (POI). As an example, evolved green fluorescent protein (EGFP) was used as the POI. Without shield-1, no binding between SsrA and SspB occurs, and EGFP is found throughout the cell. Addition of shield-1 uncages SsrA from CAPs and allow it to bind with SspB-EGFP, thereby translocating EGFP to the membrane. As shown by FIG. 9b, under no-shield-1 conditions, EGFP was localized in the cytosol and nucleus with no apparent membrane pattern as predicted. Upon adding shield-1, EGFP translocation to the plasma membrane was observed within seconds.

These results indicate that shield-1 can easily permeabilize the cell membrane and open CAPs within seconds, indicating that the CAPs-shield-1 system is useful for experiments that require rapid temporal control. These results further demonstrate that the CAPs system retains its functionality when directly transferred from yeast surface to mammalian cell culture, with no need of re-optimization. By directing CAPs-SsrA to different subcellular compartments with appropriate localization signals, POI may be recruited to various locations of interest in a shield-1-dependent manner.

### Example 4—CAPs Delocalize Proteins from the Plasma Membrane to the Cytosol in HEK 293T Cells

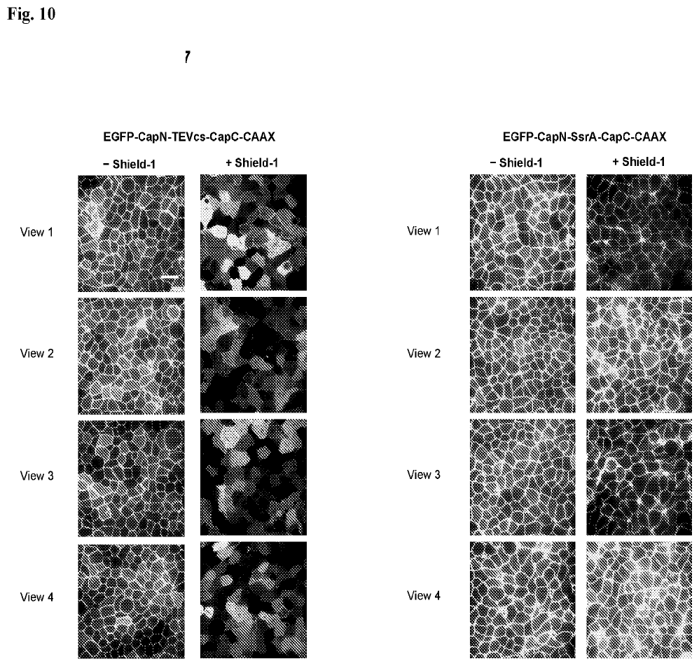

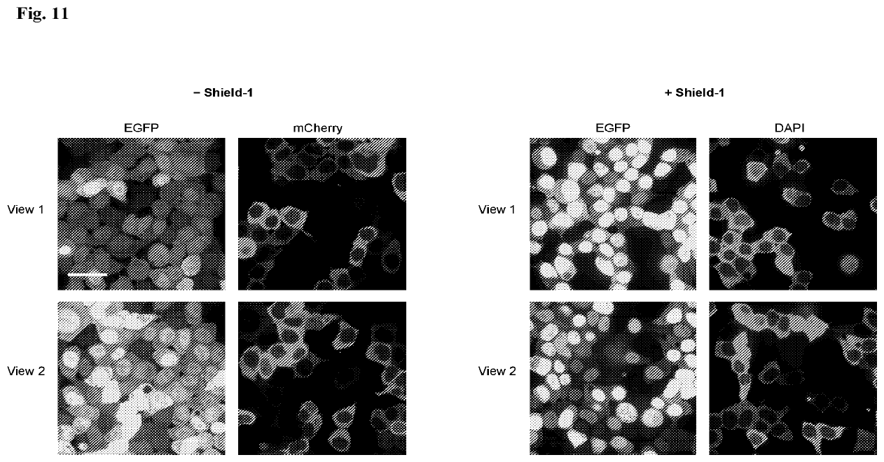

A further design shows the reverse process of membrane localization i.e., to remove a POI from the plasma membrane of use, for example, in temporally-controlled perturbation of proteins that function when localized on the plasma membrane. CAPs-caged TEVcs were anchored to the plasma membrane, followed by the POI (FIG. 9c). Under no shield-1 conditions, TEVcs are inaccessible to the co-expressed TEV protease, and the POI remains localized on the plasma membrane. With shield-1, TEVcs are uncaged and cleaved by TEV protease, thereby releasing the POI from the cell membrane. FIG. 9d and FIG. 10 show that prior to shield-1 addition, the example POI, EGFP, was almost exclusively bound to the plasma membrane. Adding shield-1 depleted the EGFP from the plasma membrane and significantly increased its presence in the cytoplasm.

Accordingly, this system delocalize a POI from its membrane location to cytoplasm location in a shield-i-dependent manner. Similar to CAPs-caged SsrA, when directly transferred from the yeast surface to mammalian cell culture, CAPs were capable of caging TEVcs efficiently. The modification needed was to change the P1′ position (the last amino acid) on TEVcs from the canonical glycine to a less active methionine to reduce unwanted cleavage, as TEV protease is present for much longer time in this assay (2-3 days) than in the yeast surface assay (3 hours).

### Example 5—CAPs Control of the Nuclear-Cytosolic Distribution of Proteins in HEK 293T Cells

The nucleocytoplasmic distribution of many eukaryotic proteins is a common determinant of their functions³⁰. Previously, NLS has been controlled by light through customized engineering with the AsLOV2 domain^{13,14}. To test if NLS is controlled by CAPs in a shield-I-dependent manner, a single-chain construct was designed in which a POI is expressed as a fusion protein with CAPs-caged NLS (FIG. 9e). In the no shield-1 condition, NLS is sterically blocked, and the POI should be found throughout the cell. Addition of shield-1 opens the CAPs and exposes NLS to endogenous importins, bringing the POI to the nucleus. Due to the strength of the NLS, a weak nuclear export signal, PKIt¹³, was added to the construct to reduce the nuclear localization of the protein in the basal state. FIG. 9f and FIG. 11 show that the example POI, EGFP, was found both in the cytosol and the nucleus when there was no shield-1. Upon addition of shield-1, EGFP was depleted from the cytosol and preferentially localized in the nucleus with a statistically different cytosol-to-whole cell ratio than that in the basal state.

These results indicate the general applicability of CAPs: CAPs not only cage TEVcs and SsrA with which they were initially optimized, may also be transferred to other peptides, for example, NLS. Small amounts of nuclear localization in the absence of shield-1 (FIG. 9f and FIG. 10), suggests that NLS is not completely blocked by CAPs. To further eliminate a nuclear pattern in the absence of shield-1, NLS sequences with a weaker strength may be used¹³.

### Example 6—Temporally-Gated Gene Transcription in HEK 293T Cell Culture and Neuronal Culture Using CAPs

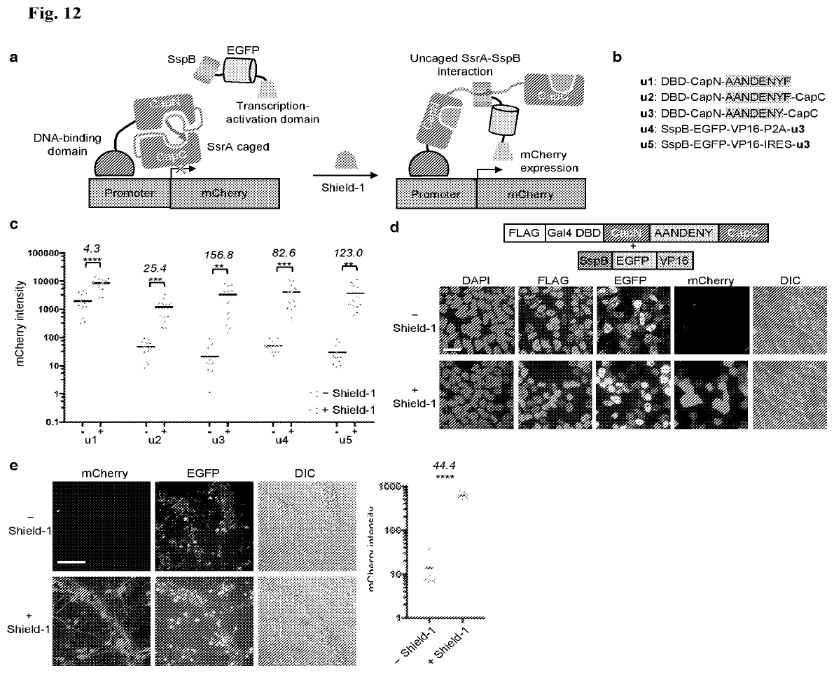

Gene transcription is a ubiquitous process in all living organisms. Temporally-controlled gene transcription provides useful means for studying the functional roles of a specific protein in living cells and animals. To test the utility of CAPs in controlling gene transcription, a split transcription factor based on the two-hybrid system” (FIG. 12a) was designed. In this design, CAPs-caged SsrA is fused to a DNA-binding domain (DBD), and SspB to a transcription-activation domain, VP16. In the presence of shield-1, SspB-VP16 is recruited to DBD-SsrA and initiates transcription of the reporter gene.

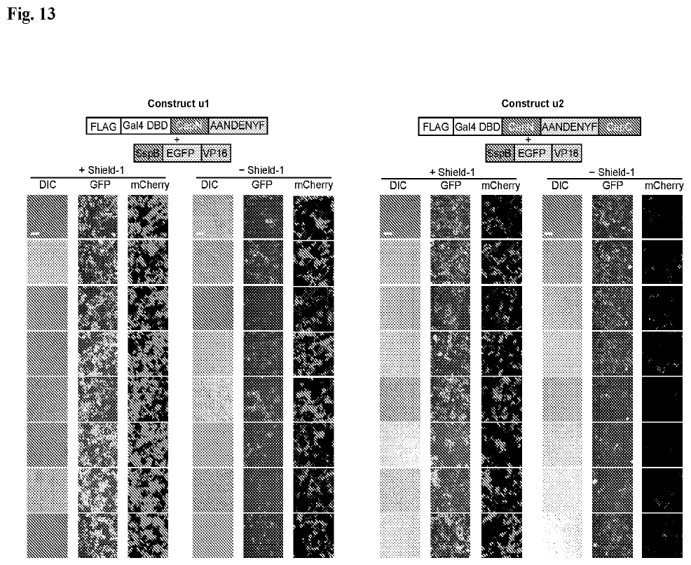

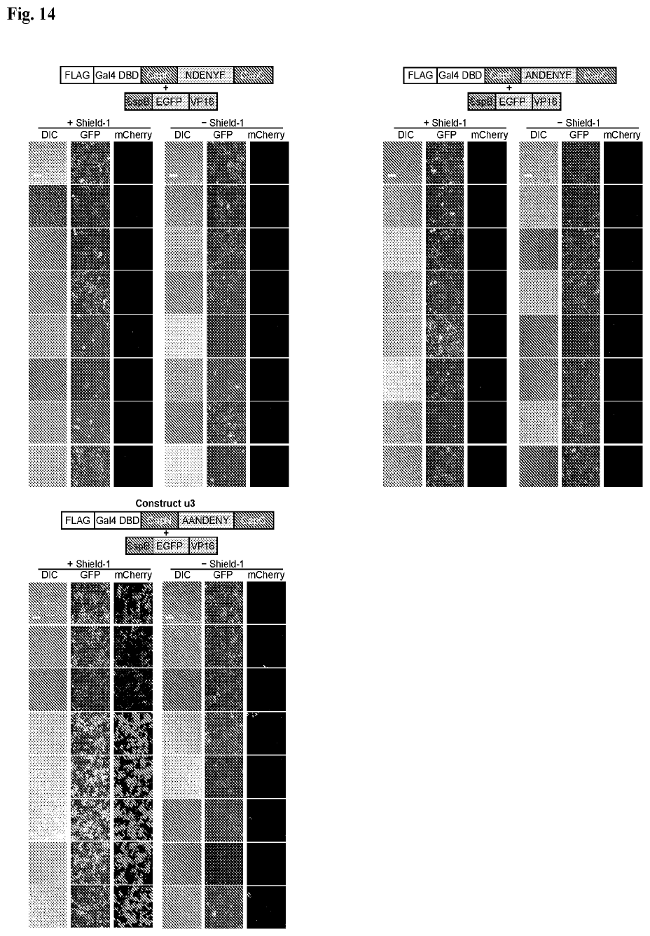

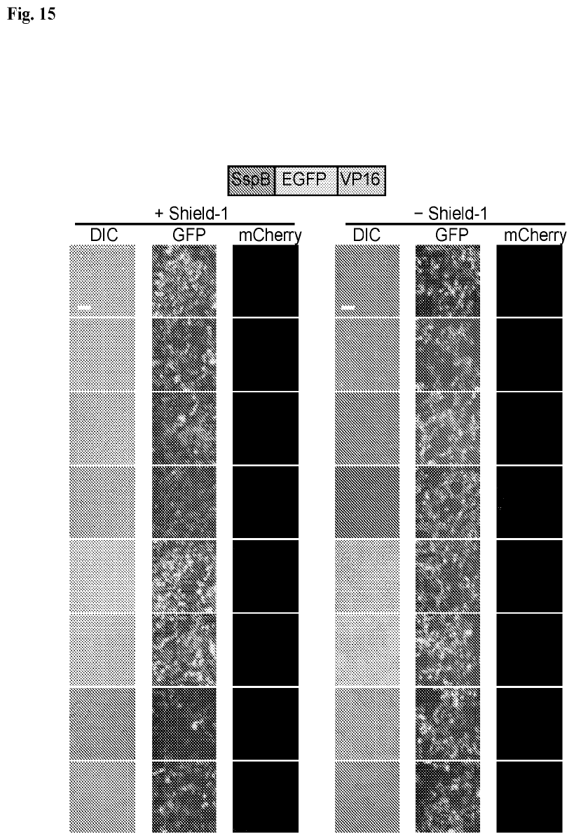

The system was tested in HEK 293T cells using the Gal4 DBD and the UAS-mCherry reporter gene. With an 8-amino-acid SsrA peptide (FIG. 12b), a ˜4-fold and ˜25-fold shield-l-induced increase in mCherry intensity was observed for CapN and CAPs caged SsrA. (FIG. 12c and FIG. 13). Tandem use of CAPs showed lower background and higher dynamic range than using CapN alone, consistent with prior observations of the CAPs-caged TEVcs on yeast surface (FIG. 8). The amino acid sequence of SsrA is tunable^{1,16}, To further lower the background because transcriptional systems are highly sensitive and often practically useful when undesired transcription is reduced to the minimum, we adjusted the SsrA sequence from both the N- and C-termini (FIG. 12b and FIG. 14) to test for more efficient caging. A 7-amino-acid SsrA sequence (AANDENY) (FIG. 12d and FIG. 14), showed a 156-fold shield-1 dependent reporter gene expression change, with 2-fold lower background and 6-fold higher signal-to-noise ratio (FIG. 12c) than the original sequence tested. The expression level of SspB-VP16 indicated by the EGFP signal was positively correlated to the amount of shield-1 added and to reporter gene expression. Conditions with higher shield-1 concentration and higher level of reporter gene expression also showed higher EGFP signal. Because shield-1 added to the EGFP-SspB-VP16 alone did not increase the level of EGFP (FIG. 15), these results indicate that the SsrA-SspB interaction stabilized the SspB protein, and non-interacting SspB was degraded.

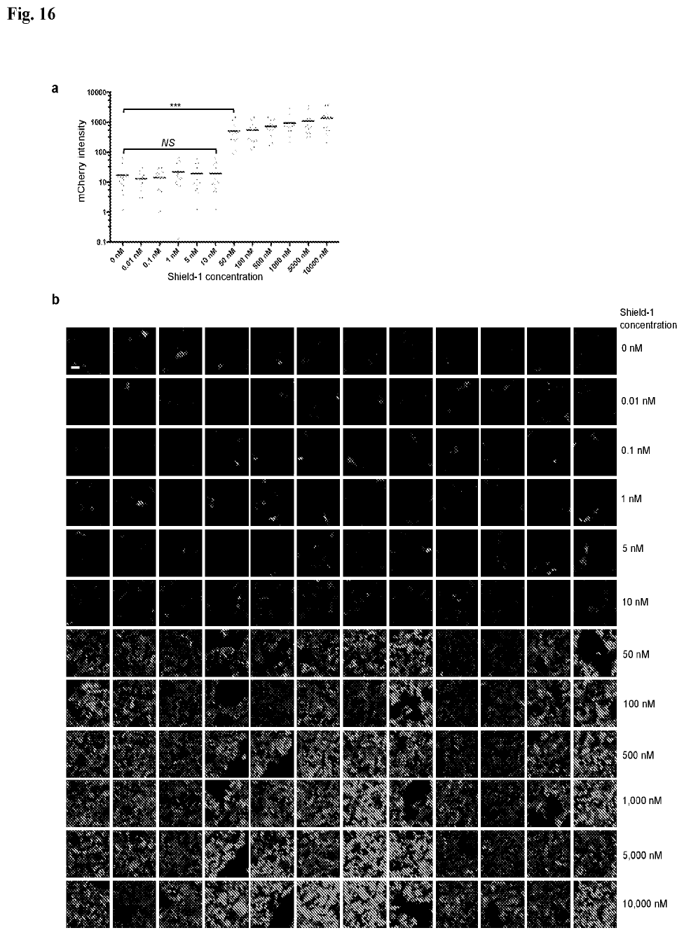

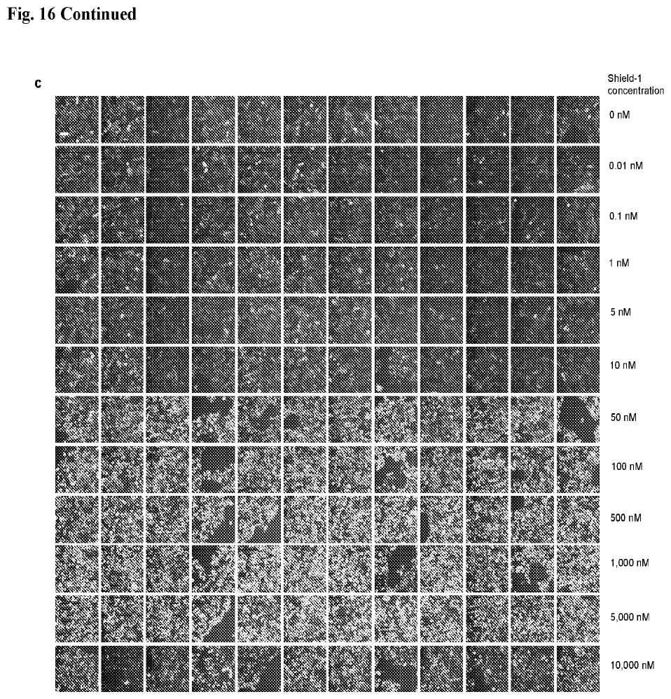

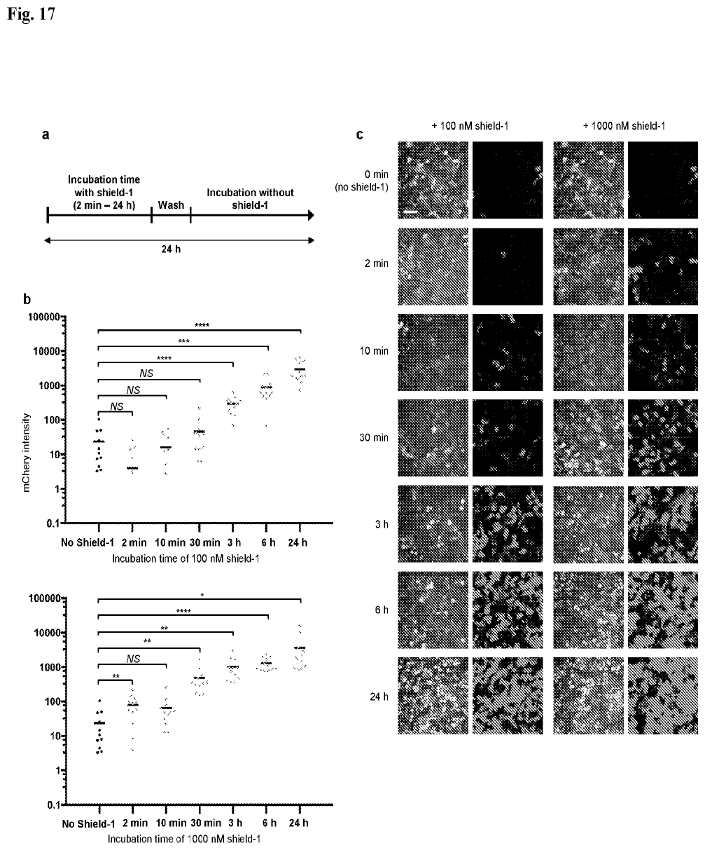

The shield-1 concentration and shield-1 incubation time required to induce robust gene expression were tested. With overnight incubation, a shield-1 concentration below 10 nM did not induce gene expression, while 50 nM or above induced >30-fold gene expression (FIG. 16). Consistent with the results above, although all conditions were transduced with the same amount of virus, those with higher shield-1 concentrations also showed higher EGFP signal, in keeping with a stabilization effect of the SspB-SsrA heterodimerization. For shield-1 incubation time, an incubation period as short as 30 min was sufficient to induce gene expression at 1 μM shield-1 concentration, but longer incubation time, on the order of hours, was required for more robust gene expression (FIG. 17).

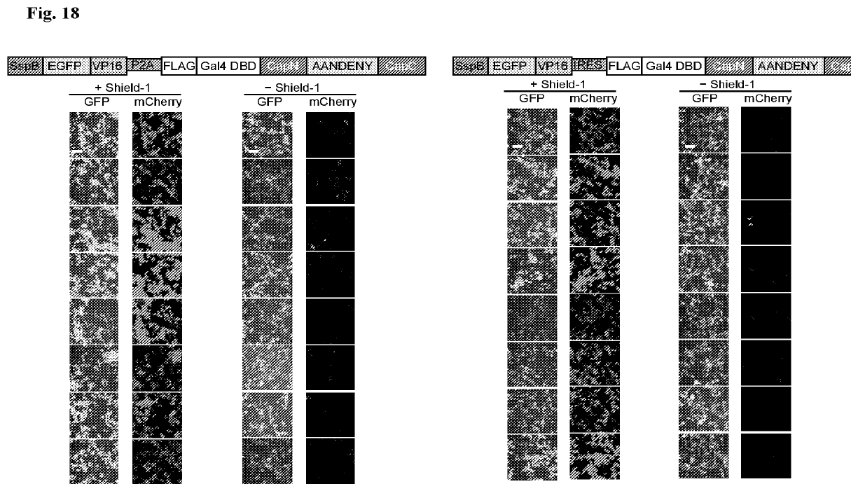

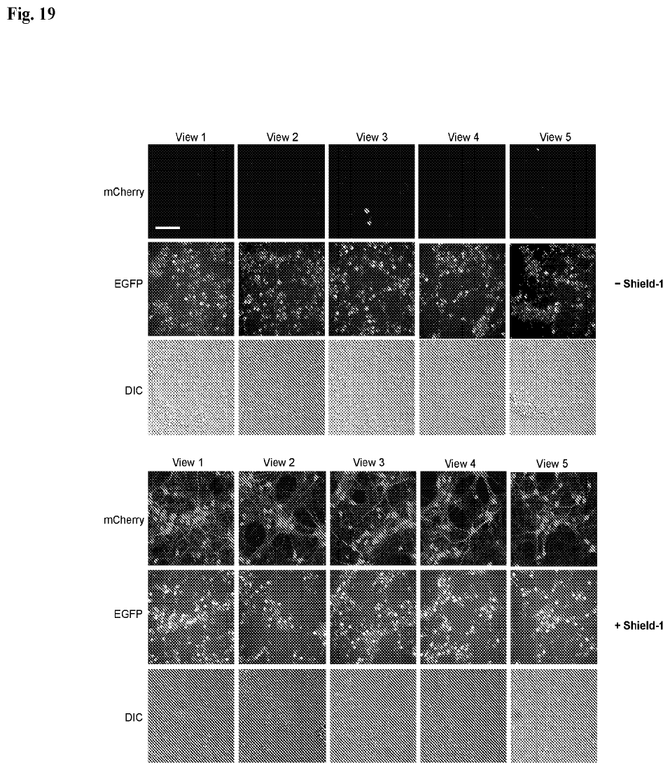

To test the CAPs chemical-dependent transcriptional system in neuroscience applications, it was tested in cultured neurons. To enable homogeneous expression of DNA in stringent experiments, we generated single viral constructs that express both the DNA-binding and the transcription-activation domains via the self-cleaving peptide P2A or the internal ribosome entry site (TRES) were constructed (FIG. 12b). Upon adding shield-1, the P2A and TRES constructs showed 83- and 123-fold increase in mCherry expression, respectively, which are comparable to the two-component system (FIG. 12c and FIG. 18). The P2A construct was then used to introduce the TetR DBD and VP16 transcription-activation domain into cultured rat cortical neurons through adeno-associated viruses (AAV), together with another AAV encoding the TRE-mCherry reporter gene. Shield-1 induced 44-fold increase in mCherry reporter gene expression compared to the no shield-1 condition (FIG. 12e and FIG. 19), showing that this system works robustly in cultured neurons.

### Example 7—Controlled Gene Transcription in Living Animals Using CAPs

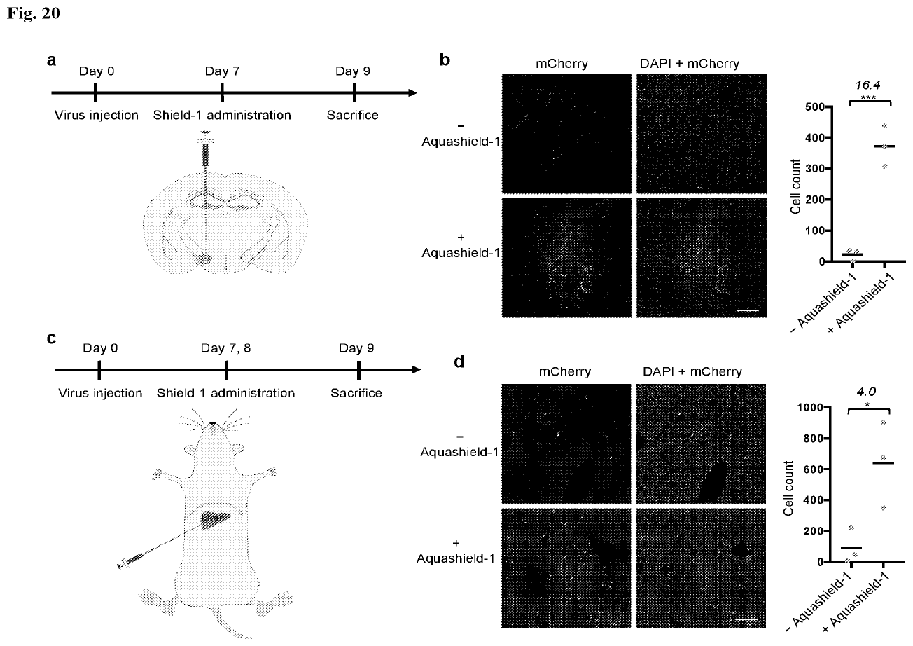

To test chemical-dependent peptide regulation in living organisms, the CAPs system was applied in mouse brain and liver. For the application in brain, stereotactic injection of AAV encoding shield-I-dependent gene regulation constructs into the lateral hypothalamic area (LHA) was performed. Seven days after viral delivery, aquashield-1 (a water-soluble analogue of the shield-1 molecule) was locally administered into LHA. Forty-eight hours after the treatment with aquashield-1 or saline, the mice were euthanized and perfused, and brain tissues were processed for analysis (FIG. 20a). In the saline treated control brains (− aquashield-1), only a few sparse neurons with mCherry expression were observed throughout the entire LHA region (FIG. 20b). In contrast, mCherry was observed in a large cluster of LHA neurons in the aquashield-1 treated brains (+ aquashield-1), at greater than 16-fold over the control (FIG. 20b) indicating shield-1 dependent gene expression.

To test the application of the CAPs chemical-dependent gene regulation system beyond neuronal tissues, AAVs encoding the CAPs constructs were injected into mouse liver. On the 7^{th} and 8^{th} day after viral delivery, aquashield-1 or saline were administered twice via intraperitoneal injection (FIG. 20c). Two days after the first aquashield-1 injection, the liver tissues were harvested for analysis. In the control mice injected with saline, few cells express mCherry in the whole liver, whereas mCherry-expressing cells were greatly increased in the liver from the aquashield-1 treated animals (FIG. 20d) indicating that systematic injection of aquashield-1 controls CAPs in mouse liver. These results show the advantages of using a small molecule to control peptide activity in comparison to light. Light is confined by its illumination area and is difficult to be applied globally in living animals. In contract, injection brings a small molecule to the whole body of the animal, thereby allowing global control of peptide activities in diverse organs in the body. Taken together, these results indicate that CAPs aquashield-1-induced gene regulation works in multiple organs in living organisms including brain and liver. In turn, aquashield-1 is readily administered through nonparenteral injection to activate gene transcription in animal tissue of interest. Compared to light-induced gene transcription systems these features of CAPs provide global control with less disturbance to the animals.

### Example 8—CapC Regulation of Opioid Peptide Met-Enkephalin

To detect CapC opioid receptor activation, a split NanoLuc assay was developed by fusing one split half of the assay to the opioid receptor, and the other half to a Gi-mimic nanobody, Nb44. Opioid receptor activation leads to recruitment of Nb44 to the receptor and reconstitution of the split NanoLuc. A cyclic adenosine monophosphate (cAMP)-dependent luciferase (GloSensor) was used to monitor the activation of mu-opioid receptor. By expressing the real-time sensor GloSensor in cells, greater luminescence is observed when the cAMP concentration is high, and lower luminescence when the cAMP concentration is low. The GloSensor assay complements the split NanoLuc assay because it measures a downstream event rather than just binding between a G protein and the receptor i.e., membrane tethered opioid peptides (M-PROBE) activation not only causes G protein recruitment, but also downstream signaling events.

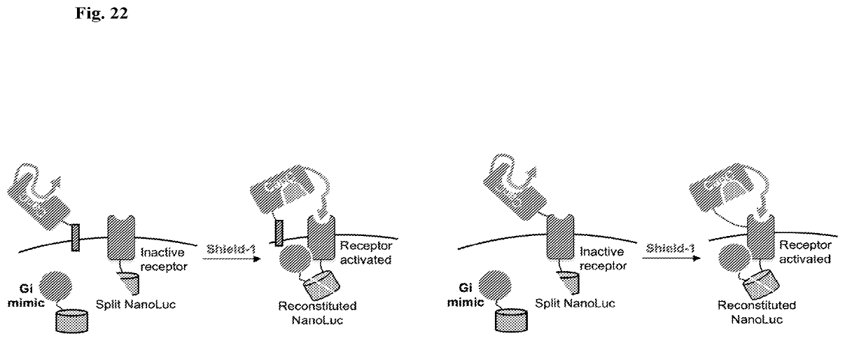

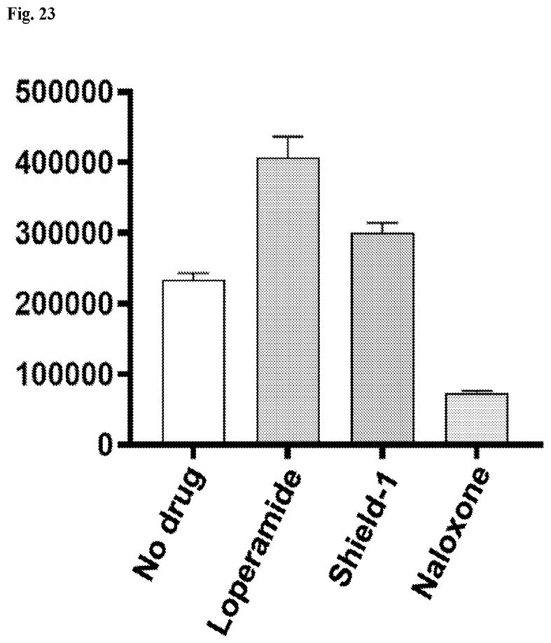

To test shield-1 chemical-dependent regulation of an opioid peptide, CapC-caged met-enkephalin was fused directly onto the N-terminus of the opioid receptor as shown in the right panel of FIG. 22. In this design, the M-PROBE cell membrane tethered opioid peptides (M-PROBE) and the opioid receptors were expressed in a fixed ratio in close proximity. FIG. 23 shows use of shield-1 dependence to control the activity of an opioid receptor through a chemically-gated membrane-tethered opioid peptide in HEK293T cells. The antagonist “Naloxone” condition provides a basal condition in the absence of receptor activation. The “No drug” condition had no external drug molecules added but did have a CapC-caged met-enkephalin on the cell surface, and its signal arose from the basal activity (i.e., background or leakage) of the M-PROBE. Loperamide is a full agonist at the mu-opioid receptor (MOR). The “Loperamide” condition showed the highest signal. Both shield-1 and loperamide increase the signal by either uncaging the opioid peptide (i.e., met-enkephalin) from CapC (i.e., shield-i-induced met-enkephalin release increased receptor activation), or by directly action of loperamide on MOR.

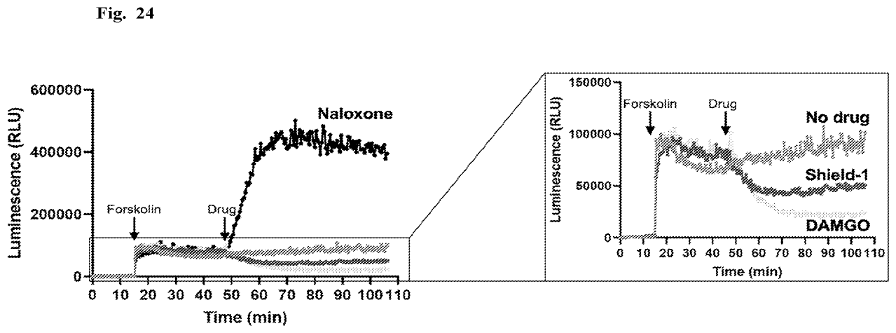

To test whether the M-PROBE induces downstream signaling as a free opioid, and that function of met-enkephalin is well-preserved in the membrane-tethered geometry, the same single chain met-enkephalin-CapC-MOR was expressed on the membrane of HEK 293T cells, and co-transfected cells with the cAMP-dependent luciferase. After overnight protein expression, HEK 293T cells were stimulated with forskolin to enhance the cAMP concentration within the cells. MOR is an inhibitory G-protein coupled receptor (GPCR), and its activation leads to decreased cAMP levels. A high cAMP concentration magnifies the effect of MOR activation. FIG. 24 shows that upon forskolin stimulation, cells exhibit significant enhancement in luminescence signals. The cells were treated under four different conditions: no drug, shield-1, MOR agonist [D-Ala², N-MePhe⁴, Gly-ol]-enkephalin (DAMGO), or MOR antagonist naloxone. Cells with no treatment showed no change in their luminescence level. Cells treated with a high concentration (10 micromolar) of DAMGO showed a large decrease (˜80%) in luminescence. The luminescence level of shield-I-treated cells fell between these 2 conditions, with a ˜50% decrease in luminescence. Cells treated with a high concentration of naloxone (10 micromolar) showed a sharp increase luminescence signal. The increase was even larger than treating cells with 1 micromolar of forskolin. These results show that in both a binding assay and a downstream cAMP assay CapC controls met-enkephalin to activate MOR in a drug-dependent manner, thereby providing methods, compositions, kits and systems to regulate target endogenous opioid receptors in specific cell populations or neuronal circuits in the central nervous system.

### Experimental Methods

### Cloning

Constructs for yeast surface display were cloned into the pCTCON2 vector. Constructs for protein expression in HEK 293T cells were cloned into the pAAV viral vector for transfection, or the pLX208 lentiviral vector for transduction. Constructs for protein expression in neuronal culture, mouse brain, and mouse liver were cloned into the pAAV vector.

FKBP for CapN was amplified from YFP-LID (Addgene plasmid #31767, Thomas Wandless laboratory). Codon optimized FKBP for CapC was synthesized by IDT.

For cloning, PCR fragments were amplified using Q5 or Taq DNA polymerase (New England Biolabs (NEB)). The vectors were double-digested with restriction enzymes (NEB), gel purified, and ligated to gel-purified PCR fragments by T4 ligation, Gibson assembly, or the In-Fusion HD Cloning Plus kit (Takara Bio). Ligated plasmid products were introduced into competent XL1-Blue E. coli cells by heat shock transformation, or in the case of In-Fusion cloning, into the Stellar competent E. coli cells from the kit following the corresponding protocol. For In-Fusion cloning, a modified protocol that proportionally decreased the amount of each reagent or competent cell by half or up to three quarters than the recommended amount was used.

### Expression and Purification of TEV Protease

Full-length TEV protease (TEVp, S219V) was expressed as a fusion to maltose binding protein (MBP) with a polyhistidine-tag. His-tag-MBP-TEVp(S219V) in a pYFJ16 vector was introduced into competent BL21-CodonPlus (DE3)-RIPL E. coli cells by heat shock transformation. Cells were cultured in 5 mL Miller's LB medium (Bio Basic) supplemented with 100 mg/L ampicillin at 37° C. with shaking at 220 r.p.m. for 6 h. Then, this saturated culture was transferred to 500 mL LB with 100 mg/L ampicillin, which was grown at 37° C. with shaking at 220 r.p.m. for roughly 2-3 h until OD₆₀₀=0.4-0.8. IPTG (isopropyl β-D-1-thiogalactopyranoside, EMD Millipore) was added to the culture to a final concentration of 1 mM, and the culture was grown at 16° C. with shaking at 220 rpm overnight. The following procedures were done at 4° C. unless otherwise specified. Cells were harvested by centrifugation at 5,000 rpm for 5 min. The cell pellet was lysed and resuspended with 15 mL ice-cold B-PER bacterial protein extraction reagent (Thermo Fisher Scientific). DTT (Fisher, freshly made) was supplemented to a final concentration of 1 mM. Benzonase nuclease (Millipore-Sigma) was added to a final concentration of ˜100 units/ml. The mixture was incubated on ice for 5 min, and centrifuged at 10,000 r.p.m. for 15 min. The supernatant was incubated with 3 mL Ni-NTA resin (Thermo Fisher Scientific) for 10 min with rotation and then transferred to a gravity column. The resin was washed with 5 mL washing buffer (30 mM imidazole, 50 mM Tris, 300 mM NaCl, 1 mM DTT, pH=7.8), then protein was eluted with 3 mL elution buffer (200 mM imidazole, 50 mM Tris, 300 mM NaCl, 1 mM DTT, pH=7.8). The eluent was concentrated with a 15 mL 10,000 Da cutoff centrifugal unit (Millipore), flash frozen in liquid nitrogen, and stored at −80° C. To obtain effective TEVp, each batch of TEVp was concentrated by at least 10-fold. With tracking of batch-to-batch variation in TEVp yield and activity.

### Expression and Purification of SspB-APEX2

SspB-APEX2 in pYFJ16 vector was expressed with polyhistidine-tag in competent BL21 E. coli cells as in the expression of TEV protease described above (under “Expression and purification of TEV protease”), except that DTT was not added into the cell lysate, washing buffer, or elution buffer. No protein concentration was performed.

### Yeast Strain and Non-Library Culture

Non-library yeast culture was generated by chemical transformation of the yeast surface display plasmid pCTCON2 into Saccharomyces cerevisiae strain EBY100 competent cells. Preparation of EBY100 competent cells has been described³⁹. To transform, 1 g of the plasmid DNA was mixed with 5 μL competent cells. 200 μL of Frozen-EZ Yeast Solution 3 (Zymo Research) was added and thoroughly mixed. The mixture was incubated at 30° C. for 30 min to 2 h, and then transferred to 5 mL SDCAA (synthetic dextrose plus casein amino acid media, 2% dextrose, 0.67% yeast nitrogen base without amino acids (BD Difco), 0.5% Casamino acids (BD Difco), 0.54% Disodium phosphate, 0.856% Monosodium phosphate) lacking tryptophan for growth at 30° C. with shaking at 220 rpm. After the initial saturation (OD₆₀₀>10) in 2-3 d, the yeast culture was passaged at least once prior to experiment, or was stored at 4° C. for up to half a year. Passaging was done by adding 500 μL of saturated culture into 5 mL fresh SDCAA media, and growing at 30° C. and 220 rpm. overnight. A negative control with no plasmid DNA accompanied each batch of transformation to ensure that the media was selective.

### Yeast Library Generation

CapN and CapC mutant libraries were generated by first producing plasmid libraries using targeted mutagenesis followed by transformation into EBY100 yeast competent cells by electroporation. Targeted mutagenesis was done by regular PCR (polymerase chain reaction) using primers with mixed bases (IDT). The first two bases in each codon corresponding to a mutant amino acid were designed to be an equal mix of A, C, G, T, and the third base was an equal mix of G and T. The PCR fragment was amplified such that it had 40 extra bases beyond the two restriction sites used for linearizing the vector.

500 ng of the template DNA plasmid (Aga2p-FLAG-FKBP-binder sequence-TEVcs-HA) was mixed with 100 mol forward and reverse primers annealing outside of the FKBP gene, 1×Q5 High GC Enhancer, 1×Q5 Reaction Buffer, 1 unit of Q5 High-Fidelity Polymerase, 10 nmol dNTP (VWR) in a total volume of 50 μL (two reaction for each library).
CapN libraries:
Forward primer:
All libraries:

SEQ ID. NO: 9
GGCTCTGGTGCTAGCGACTACAAGGATGACGACGATAAGACTAGT
Reverse primer:
Library 1:
SEQ ID NO: 10
GGATCCACCCTGGAAGTAGAGATTTTCMNNMNNMNNMNNMNNMNNCGCC
ACTTCCTCCACTCCACGC
Library 2:
SEQ ID NO: 11
GGATCCACCCTGGAAGTAGAGATTTTCMNNMNNMNNMNNMNNMNNMNNC
GCCACTTCCTCCACTCCACGC
Library 3:
SEQ ID NO: 12
GGATCCACCCTGGAAGTAGAGATTTTCMNNMNNMNNMNNMNNMNNMNNM
NNCGCCACTTCCTCCACTCCACGC
Library 4:
SEQ ID NO: 13
GGATCCACCCTGGAAGTAGAGATTTTCMNNMNNMNNMNNMNNMNNMNNM
NNMNNCGCCACTTCCTCCACTCCACGC

The PCR was run for 20 cycles with annealing temperature of 60° C. The product DNA was gel-purified, and amplified by the following primers:
Forward primer:

All libraries:
SEQ ID NO: 14
GGCTCTGGTGCTAGCGACTACAAGGATGACGACGATAAGACTAGT
Reverse primer:
All libraries:
SEQ ID NO: 15
CTCGAGCTATTAAGCGTAATCTGGAACGTCATATGGGTAGGATCCACCC
TGGAAGTAGAGATTTTC

CapC libraries:
Forward primer:

Library 1:
SEQ ID NO: 16
GCTAGCGCAGCGAATGATGAAAATTACTTCNNKNNKNNKCCTAATTTGN
NKNNKNNKGGATCAGGCGGTTCTGGTACTG
Library 2:
SEQ ID NO: 17
GCTAGCGCAGCGAATGATGAAAATTACTTCNNKNNKCCTAATTTGNNKN
NKNNKNNKTCAGGCGGTTCTGGTACTGG
Library 3:
SEQ ID NO: 18
CTGCAGCAAGGTCTGCAGG
Reverse primer:
Library 1:
SEQ ID NO: 19
TCAGATCTCGAGCTATTACTTATCGTCGTC
Library 2:
SEQ ID NO: 20
TCAGATCTCGAGCTATTACTTATCGTCGTC
Library 3:
SEQ ID NO: 21
GAGATGGTTTCCACCTGCACTCCMNNMNNMNNMNNMNNMNNTCCAGTAC
CAGAACCGCCTG

The PCR was run for 20 cycles with annealing temperature of 60° C. The product DNA was gel-purified, and amplified by the following primers:
Forward primer:

Library 1:
SEQ ID NO: 22
CTGCAGCAAGGTCTGCAGGCTAGTGGTGGAGGAGGCTCTGGTGCTAGCG
CAGCGAATGATGAAAATTACTTC
Library 2:
SEQ ID NO: 23
CTGCAGCAAGGTCTGCAGGCTAGTGGTGGAGGAGGCTCTGGTGCTAGCG
CAGCGAATGATGAAAATTACTTC
Library 3:
SEQ ID NO: 24
GGAGTGCAGGTGGAAACCATCTC

Reverse primer:

All libraries:
SEQ ID NO: 25
TCAGATCTCGAGCTATTACTTATCGTCGTC

The template DNA was equally divided into 8 portions. Each portion was mixed with 100 μM forward and reverse primers, 1×Taq Reaction Buffer without magnesium chloride, 2 mM magnesium chloride, 2 units of Taq Polymerase, 10 nmol dNTP (VWR) in a total volume of 50 μL. For the same library, 8 PCRs were gel-purified and combined.

Linearized vector was gel purified. We combined 2 μg of linearized vector with 8 g of PCR fragment and concentrated using pellet paint (Millipore) following manufacturer's protocols on the day of electroporation. The precipitated DNA was resuspended in 20 μL of ultra-pure water (Thermo Fisher Scientific).

In parallel, fresh electrocompetent EBY100 yeast cells were prepared. Cells were passaged at least twice in YPD (yeast extract peptone dextrose media, 20 g dextrose, 20 g peptone and 10 g yeast extract in 1 L deionized water) prior to this procedure to ensure that cells were healthy. Saturated culture of yeast was inoculated to 200 mL of YPD to an initial OD₆₀₀ of 0.3-0.4. Cells were grown at 30° C. with shaking at 220 rpm. for roughly 6 h until OD₆₀₀ reached 1.8-2.2, and then centrifuged at 4° C. and 3,000 rpm for 3 min. The cell pellet was resuspended and washed with ice-cold water, centrifuged again, and resuspended in 50 mL ice-cold sterile lithium acetate (100 mM in water). After this step, yeast was placed on ice until after electroporation. DTT was added to a final concentration of 10 mM. The cells were then incubated at 30° C. and 220 rpm for 20 min, centrifuged at 4° C. and 3,000 rpm for 3 min, washed with 50 mL ice-cold water, centrifuged again, and resuspended in 0.8 mL of electroporation buffer (1M sorbital/1 mM CaCl2)).

Immediately after yeast cells preparation, 400 μL of the cells was mixed with the 20 μL concentrated DNA prepared as described above, and the mixture was transferred to an electroporation cuvette. Electroporation was done using a Bio-Rad Gene pulser XCell with the following settings: 500 V, 15 msec pulse duration, one pulse only, 2 mm cuvette. Cells were immediately rescued with 1 mL of 1:1 mixture of sorbitol and YPD media. The cuvette was washed 3 times, each time with 1 mL of the fresh sorbitol and YPD mixture. All cells were combined and incubated at 30° C. for 30 min with no shaking, then 30 min with shaking at 220 rpm 10 μL of the cells was plated onto three SDCAA plates with a serial dilution of 100×, 1,000×, and 10,000×, for determining library size. The rest of the cells was centrifuged at 3,000 rpm for 2 min, resuspended in 5 mL SDCAA media, centrifuged again, and transferred to 200 mL SDCAA media supplemented with 1% Penicillin-Streptomycin (50 units/mL penicillin and 50 g/mL streptomycin, Gibco), and 30 μg/mL kanamycin (DOT Scientific). The culture was grown at 30° C. with shaking at 220 rpm for 12-24 h until OD₆₀₀ reached 15 (OD₆₀₀˜1 corresponds to roughly 1×10⁷ yeast cells/mL). For each batch of library generation, a negative control was included with no plasmid DNA.

The combined library size for CapN and CapC (four libraries combined for CapN, 3 libraries combined for CapC) was each determined to be ˜1×10⁷.

### Yeast Labeling

The labeling procedures were used for both non-library yeast culture and yeast library culture. (Generation of non-library and library yeast culture is described above under “Yeast strain and non-library culture” and “Yeast library generation”, respectively.) Yeast was freshly passaged in SDCAA media prior to experiment. To induce expression of the pCTCON2 plasmid, 500 μL of the overnight yeast in SDCAA media was added to 5 mL of SGCAA (synthetic galactose plus casein amino acid media, 2% galactose, 0.67% yeast nitrogen base without amino acids (BD Difco), 0.5% Casamino acids (BD Difco), 0.54% Disodium phosphate, 0.856% Monosodium phosphate)) media and grown at 30° C. with shaking at 220 rpm. overnight. Prior to labeling, 250 μL (or 1 mL for the first round of library selection) overnight yeast in SGCAA media was centrifuged at 8000 rpm for 30 s, and the supernatant was discarded. The cell pellet was resuspended and washed twice, each time with 1 mL of PBSB (sterile phosphate-buffered saline supplemented with 0.1% bovine serum albumin).

Yeast that expressed TEV protease cleavage site (CapN constructs) was treated with TEVp prior to labeling with antibody-fluorophore conjugate. Samples were incubated with 200 μL PBSB containing TEVp (expressed and purified as described above, under “Expression and purification of TEV protease”) and 5 μM shield-1 at 4° C. for 3 h with rotation. For negative control, either TEVp or shield-1, or both, was not present in PBSB. 1 mM DTT and 30 mM reduced and 3 mM oxidized of glutathione were added to all samples to maintain TEVp under reducing conditions.

Yeast that expressed SsrA (CapC constructs) was subject to APEX2 labeling⁴⁰ prior to labeling with antibody-fluorophore and streptavidin-fluorophore conjugates. Samples were incubated with 100 μL of PBSB containing SspB-APEX2 (expressed and purified as described above, under “Expression and purification of SspB-APEX2”) and 5 μM shield-1 at room temperature for 10 min with rotation. For negative controls, either SspB-APEX2 or shield-1, or both, was not present in PBSB. After incubation, samples were washed twice, each time with 1 mL PBSB, and were resuspended in 950 μL PBSB with 1% BSA (bovine serum albumin). 1 μL biotin-phenol (1 mM in dimethyl sulfoxide) was added and thoroughly mixed with the sample. Then, 1 μL of hydrogen peroxide (0.5 mM in water, freshly prepared, EMD chemicals) was added and thoroughly mixed. After incubation for exactly 2 min, 200 μL of quenching solution 1 (30 mM Trolox (Thermo Fisher Scientific), 60 mM sodium ascorbate (Millipore Sigma), freshly prepared) was added. The sample was centrifuged at 8,000 r.p.m. for 30 s, and the supernatant was discarded. 400 μL of quenching solution 2 (5 mM Trolox, 10 mM sodium ascorbate, freshly prepared) was then added to resuspend the cell pellet. After another centrifugation at 8,000 rpm for 30 s, the supernatant was discarded, and the sample was washed twice, each time with 1 mL PBSB.

After TEVp or APEX2 labeling, samples were labeled with antibody-fluorophore and/or streptavidin-fluorophore conjugates. To label FLAG and HA epitope tags, primary anti-FLAG or anti-HA antibodies were used, followed by secondary antibodies conjugated with Alexa Fluor 568 or 647. To detect biotinylated proteins from APEX2 labeling, streptavidin conjugated with PE (phycoerythrin) was used. All antibodies were diluted to 1 μg/mL in PBSB, streptavidin-PE (Jackson Immuno Research) was diluted 200-fold, and each yeast sample was incubated with 100 μL of the mixture at room temperature for 15 min with rotation. Two washes with PBSB were done after each step of labeling. All samples were resuspended in PBSB and analyzed or sorted by FACS (fluorescence-activated cell sorting, following procedures described under “FACS analysis and library selection”) within 24 h of labeling.

### FACS Analysis and Library Selection

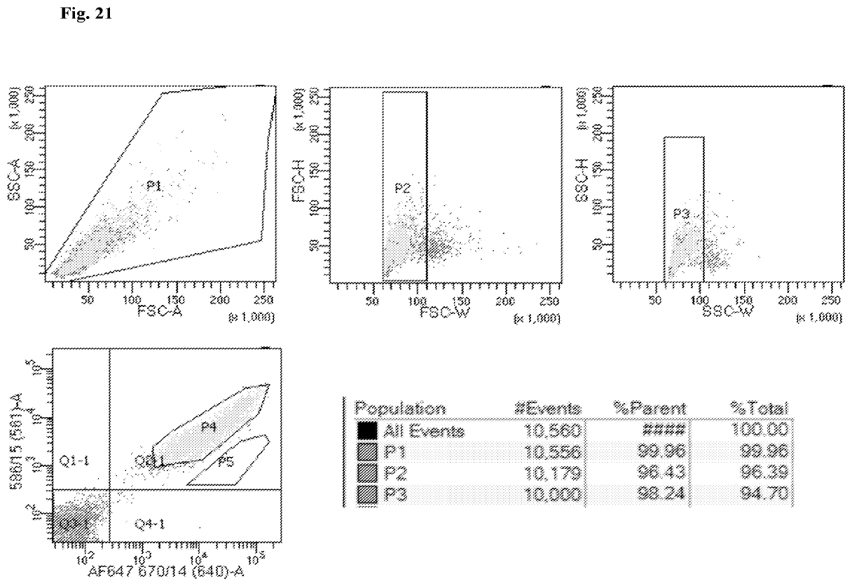

After labeling according to the procedures described above (under “Yeast labeling”), non-library yeast samples were analyzed with an LSRFortessa cell analyzer flow cytometer (BD Biosciences) equipped with 640 nm laser and 670/14 emission filter (for Alexa Fluor647) as well as 561 nm laser and 586/15 emission filter (for Alexa Fluor568 and PE). Library samples were sorted with a FACSAria III cell sorter flow cytometer (BD Biosciences) equipped with 633 nm laser and 660/20 emission filter (for Alexa Fluor647) as well as 561 nm laser and 582/15 emission filter (for Alexa Fluor568 and PE). Single yeast cells were selected by consecutive gates P1 through according to FIG. 21 and further analyzed.

A CapN library was generated and labeled as described above (under “Yeast library generation” and “Yeast labeling”, respectively). For positive selection, both TEVp and shield-1 were added. For negative selection, only TEVp but not shield-1 was added. A total of 4 rounds of selections were performed. The number of cells collected for each round was as follows:

### Round 1 (Negative Selection):

0.034% of the cells were collected from Library 1 (1.4×10⁷ cells)
0.041% of the cells were collected from Library 2 (2.2×10⁷ cells)
0.028% of the cells were collected from Library 3 (2.1×10⁷ cells)
0.025% of the cells were collected from Library 4 (1.0×10⁷ cells)

The collected cells are combined for further selection.

### Round 2 (Positive Selection):

3.4% of the cells were collected (1.2×10⁶ cells)

### Round 3 (Negative Selection):

11.3% of the cells were collected (3.5×10⁶ cells)

### Round 4 (Positive Selection):

5.8% of the cells were collected (1.5×10⁶ cells)

A CapC library was generated and labeled as described above (under “Yeast library generation” and “Yeast labeling”, respectively). For positive selection, both SspB-APEX2 and shield-1 were added. For negative selection, only SspB-APEX2 but not shield-1 was added. A total of 2 rounds of selections were performed. The number of cells collected for each round was as follows:

### Round 1 (Negative Selection):

0.3% of the cells were collected (1.5×10⁴ cells)

### Round 2 (Positive Selection):

0.1% of cells collected (3×10³ cells)

All yeast cells were collected into 5 mL SDCAA media with 100 units/mL penicillin, 100 μg/mL streptomycin, and 30 μg/mL kanamycin. Immediately after sorting, cells were grown at 30° C. with shaking at 220 rpm for 2-5 d until saturation. For the next round of sorting, cells were passaged at least once in SDCAA media and labeled according to the procedures described above, under “Yeast labeling”. After the last round of sorting, plasmids were extracted by Zymoprep Yeast Plasmid Miniprep II kit (Zymo Research) with modified manufacturer's protocol. Overnight yeast culture (500 μL) was transferred to 10 mL fresh SDCAA media and grew until OD₆₀₀=1-2. Yeast cells were spun down and washed with phosphate buffered saline (PBS) once, followed by resuspension with 200 μL Solution I and 6 μL Zymolase solution provided in the kit. Vigorous vortexing was performed for >1 minute. Yeast cells were placed in the 37° C. shaker overnight to degrade the cell wall. Following the overnight incubation, yeast cells were vigorously vortexed for >5 minutes. Then, 200 μL Solution II was added to the yeast cells, followed by brief vortexing and incubation at room temperature for 5 min. 400 μL neutralizing solution was added and the cells were vortexed briefly. The cell lysate was spun down at 20,000×g for 10 min and the supernatant was loaded to a DNA column (Epoch Life Science) to purify the plasmid DNA. The extracted plasmid DNA from the yeast library was heat shock transformed into XL1-Blue E. coli. Individual clones were sequenced, transformed into EBY100 yeast cells (as described above under “Yeast strain and non-library culture”), labeled (as described above under “Yeast labeling”), and analyzed by FACS (as described earlier in this section).

### All-Atom Molecular Dynamics Simulations

Atomistic molecular dynamics simulations of FKBP and the RYSPNL hexapeptide with capped N- and C-termini (Ac-RYSPNL-NHMe) were performed to analyze the binding pose of the peptide to its binding site. The simulations were performed with the GROMACS 2018.1 software package⁴¹ with the AMBER99SB-ILDN protein force field⁴² and TIP3P water⁴³.

The simulations based were started based on a structure of the FKBP12 protein (PDBID:INSG) in which the F36V mutation was introduced using DeepView/Swiss-Pdb Viewer⁴⁴. The peptide was placed in an alpha-helical conformation with sidechains generated with the Scwrl4 program⁴⁵ outside of the putative FKBP binding site. The initial distance of the peptide center of mass relative to the center of mass of the V36 of FKBP was 16.7 Å. The smallest distance between any atom of the peptide and the FKBP protein was 5.75 Å, which allows for a separation by at least 2 hydration layers.

The system was placed in a cubic simulation box of 75 Å×75 Å×75 Å and solvated with 13444 water molecules in addition to 67 water molecules resolved in the crystal structure. 38 sodium and 40 chloride ions were added to approximate physiological salt concentrations of 150 mM and to neutralize the charge of the protein (+1 e) and peptide (+1 e) at pH 7. The protonation states of the protein sidechains were estimated by the pdb2gmx tool in GROMACS.

The simulations were performed in periodic boundary conditions with the particle-mesh Ewald⁴⁶ algorithm for the treatment of long-ranged electrostatic interactions using a 1.2 Å grid constant and fourth order interpolation. A 10 Å cutoff was used for short-ranged Lennard-Jones and electrostatic interactions with corrections for the pressure and total energy. The LINCS algorithm⁴⁷ was used to constrain bond lengths in the protein during dynamics simulations and the SETTLE algorithm⁴⁸ was used to constrain the geometry of water molecules.

After a steepest descent energy minimization for 1000 steps, the system was equilibrated in molecular dynamics simulations in the isothermal-isobaric ensemble at 300 K and 1 bar for 100 picoseconds using a simulation time step of 1 femtosecond and a Berendsen⁴⁹ thermostat and barostat with a 1 picosecond time constant. In this equilibration, the non-hydrogen atoms of the protein and peptide were constrained to their initial positions using isotropic position restraints with force constants of 10 k/(mol Å2). This was followed by a production simulation for 2 microseconds using a time step of 2 femtoseconds without position restraints in the isothermal-isobaric ensemble at 300 K and 1 bar with a Nose-Hoover thermostat^{50,51} with a 1 picosecond time constant and a Parrinello-Rahman barostat⁵² with a time constant of 2 picoseconds.

The trajectories were analyzed using a simple clustering algorithm⁵³ using non-hydrogen atoms and a 1.5 Å cut-off Further, the distance between the center of mass for each sidechain of the peptide and the V36 of FKBP was monitored.

### Fluorescence Microscopy of Cultured Cells

Confocal imaging was performed on a Nikon inverted confocal microscope with 10× air, 20× air, and 60× oil-immersion objectives, outfitted with a Yokogawa CSU-X1 5000RPM spinning disk confocal head, and Ti2-ND-P perfect focus system 4, a compact 4-line laser source: 405 nm (100 mW) 488 nm (100 mW), 561 nm (100 mW) and 640-nm (75 mW) lasers. The following combinations of laser excitation and emission filters were used for various fluorophores: DAPI (405 nm excitation; 455/50 emission), EGFP/Alexa Fluor 488 (488 nm excitation; 525/36 emission), mCherry/Alexa Fluor 568 (568 nm excitation; 605/52 emission), Alexa Fluor 647 (647 nm excitation; 705/72 emission), and differential interference contrast (DIC). ORCA-Flash 4.0 LT+sCMOS camera. Acquisition times ranged from 100 to 1000 msec. All images were collected and processed using Nikon NIS-Elements hardware control and analysis module.

HEK 293T cell culture and transfection

Low passage HEK 293T cells (less than 20 passages) were cultured at 37° C. under 5% CO2 in T25 or T75 flasks in complete growth media, 1:1 DMEM (Dulbecco's Modified Eagle medium, Gibco): MEM (Eagle's minimal essential medium) supplemented with 10% FBS (Fetal Bovine Serum, Sigma), 50 mM HEPES (Gibco), and 10% Penicillin-Streptomycin (50 units/mL penicillin and 50 μg/mL streptomycin, Gibco). For imaging experiments, 48-well plates were pretreated with 200 μL of 20 μg/mL human fibronectin (Millipore Sigma) for 10 min at 37° C. HEK 293T cells were then plated in 48-well plates at 60%-90% confluence. A mix of DNA was incubated with 1 μL 1 mg/mL PEI max solution in 10 μL serum-free DMEM media for 15 min at room temperature. Complete DMEM growth media (100 μL) was then mixed with the DNA-PEI max solution and added to the HEK 293T cells that were fully attached to well bottom and incubated for 18 h before further processing.

### Production of Lentivirus Supernatant for HEK Cell Transduction

New cell culture flasks were incubated with 20 μg/mL human fibronectin (HFN, Millipore Sigma) at 37° C. for at least 10 min to facilitate cells to attach to the surface and increase transfection efficiency. After incubation, HFN was aspirated, and HEK293T cells were plated at 70-90% confluence. For a T25 flask, cells were grown at 37° C. for 1-3 h in 5 mL complete DMEM growth media, 1:1 DMEM (Dulbecco's Modified Eagle medium, Gibco): MEM (Eagle's minimal essential medium) supplemented with 10% FBS (Fetal Bovine Serum, Sigma), 50 mM HEPES (Gibco), and 1% Penicillin-Streptomycin (50 units/mL penicillin and 50 g/mL streptomycin, Gibco). After incubation, 2.5 g viral DNA, 0.25 g pVSVG, and 2.25 μg delta8.9 lentiviral helper plasmid were combined with 250 μL of DMEM and thoroughly mixed.

Then, 25 μL PEI max solution (polyethylenimine HCl Max, pH 7.3, 1 mg/mL, Polysciences) was added. The mixture was incubated at room temperature for at least 10 min, mixed with 1 mL complete media, and transferred to the T25 flask. Cells were incubated at 37° C. for 48 h, and the supernatant with virus was collected, flash frozen in liquid nitrogen, and stored at −80° C. for up to one year.

### HEK 293T Cell Culture and Infection

Low passage HEK 293T cells (less than 20 passages) were cultured at 37° C. under 5% CO2 in T25 or T75 flasks in complete growth media. For imaging experiments, 24-well glass-bottom plates (Cellvis) were pretreated with 350 μL 20 μg/mL human fibronectin (Millipore Sigma) for 10 min at 37° C. HEK 293T cells were then plated in 24-well plates at 40%-60% confluence. For infection of a single well in a 24-well plate, 100-200 μL of each supernatant virus was added gently to the top of the media and incubated for 48 h before further processing.

### HEK 293T Cell Stimulation, Imaging, and Data Analysis for Shield-1 Induced Protein Translocation to the Plasma Membrane

HEK 293T cells were plated in 24-well plates as described above at 80% confluence and then transfected with 200 ng of mCherry-CapN-SsrA-CapC-CAAX, and 200 ng of SspB-EGFP plasmid. SEQ ID NO: 18 comprises a representative DNA sequence comprising a CapN sequence, a SsrA sequence (bold underlined), and CapC sequence (underlined).

SEQ ID NO: 26:
GGCGTCCAAGTCGAGACAATATCTCCCGGCGACGGACGAACGTTTCCGA
AGCGGGGACAGACTTGCGTCGTTCACTACACAGGAATGCTGGAGGATGG
CAAAAAGGTCGATTCCAGCCGGGACAGGAATAAGCCGTTCAAATTCATG
CTGGGAAAACAGGAGGTAATACGAGGTTGGGAGGAGGGTGTGGCGCAAA
TGTCTGTCGGTCAGCGGGCGAAACTGACCATTTCTCCTGATTATGCTTA
CGGAGCGACAGGCCATCCGGGGATCATTCCGCCCCACGCTACCCTGGTA
TTCGATGTAGAACTCTTGAAGCTCGAAACCAGAGGGGTTGAAGAAGTAG
CCAGATATTCCCCCAACCTG [symbol] GGTACT
CCTAATTTGCGGCCTTTTGGTTCAGGCGGTTCTGGTACTGGATCTGGTT
CTGGAGGTTCTGGAGTGCAGGTGGAAACCATCTCCCCAGGAGACGGGCG
CACCTTCCCCAAGCGCGGCCAGACCTGCGTGGTGCACTACACCGGGATG
CTTGAAGATGGAAAGAAAGTTGATTCCTCCCGGGACAGAAACAAGCCCT
TTAAGTTTATGCTAGGCAAGCAGGAGGTGATCCGAGGCTGGGAAGAAGG
GGTTGCCCAGATGAGTGTGGGTCAGAGAGCCAAACTGACTATATCTCCA
GATTATGCCTATGGTGCCACTGGGCACCCAGGCATCATCCCACCACATG
CCACTCTCGTCTTCGATGTGGAGCTTCTAAAACTGGAA

SEQ ID NOs: 27-45 comprise a representative sequence comprising a CapC-caged met-enkephalin amino acid sequence expressed as a separate construct (FIG. 22, left panel) with a binding sequence for FKBP.

Protein sequence:

Surface trafficking signal peptide-
SEQ ID NO: 27
MKTIIALSYIFCLVFA
Met-enkephalin-
SEQ ID NO: 28
YGGFM
Binding sequence, linker, and FKBP binding site-
SEQ ID NO: 29
PNLRPFGSGGSGTGSGSGGSGVQVETISPGDGRTFPKRGQTCVVHYTGMLEDGKKVDSS
RDRNKPFKFMLGKQEVIRGWEEGVAQMSVGQRAKLTISPDYAYGATGHPGIIPPHATLV
FDVELLKLE
AgeI restriction site-
SEQ ID NO: 30
TG
Linker-
SEQ ID NO: 31
GGSGSGSGGSGGSGG
Truncated human cluster of differentiation 4 (CD4) transmembrane domain-
SEQ ID NO: 32
LPTWSTPVQPMALIVLGGVAGLLLFIGLGIFFCVRCRHRRR
Linker-
SEQ ID NO: 33
KGSGSTSGSGSGGSRGSGGSSGG
CIBN (truncated cryptochrome-interacting basic-helix-loop-helix protein)
for enhancing surface trafficking-
SEQ ID NO: 34
MNGAIGGDLLLNFPDMSVLERQRAHLKYLNPTFDSPLAGFFADSSMITGGEMDSYLSTA
GLNLPMMYGETTVEGDSRLSISPETTLGTGNFKAAKFDTETKDCNEAAKKMTMNRDDL
VEEGEEEKSKITEQNNGSTKSIKKMKHKAKKEENNFSNDSSKVTKELEKTDYIH
Hemagglutinin (HA) epitope tag-
SEQ ID NO: 35
YPYDVPDYA

Surface trafficking signal peptide-
SEQ ID NO: 36
ATGAAGACCATCATCGCCCTGAGCTACATCTTCTGCCTGGTGTTCGCC
Met-enkephalin-
SEQ ID NO: 37
TACGGCGGATTTATG
Binding sequence, linker, and FKBP binding site-
SEQ ID NO: 38
CCTAATTTGCGGCCTTTTGGTTCAGGCGGTTCTGGTACTGGATCTGGTTCTGGAGGTT
CTggagtgcaggtggaaaccatctccccaggagacgggcgcaccttccccaagcgcggccagacctgcgtg
gtgcactacaccgggatgcttgaagatggaaagaaagttgattcctcccgggacagaaacaagccctttaa
gtttatgctaggcaagcaggaggtgatccgaggctgggaagaaggggttgcccagatgagtgtgggtcaga
gagccaaactgactatatctccagattatgcctatggtgccactgggcacccaggcatcatcccaccacat
gccactctegtcttcgatgtggagcttctaaaactggaa
AgeI restriction site-
SEQ ID NO: 39
ACCGGT
Linker-
SEQ ID NO: 40
GGTGGAAGTGGATCAGGCAGCGGTGGATCTGGAGGTAGCGGCGGA
Truncated human cluster of differentiation 4 (CD4) transmembrane domain-
SEQ ID NO: 41
CTGCCCACATGGTCCACCCCGGTGCAGCCAATGGCCCTGATTGTGCTGGGGGGCGTC
GCCGGCCTCCTGCTTTTCATTGGGCTAGGCATCTTCTTCTGTGTCAGGTGCCGGCACC
GAAGGCGC
Linker-
SEQ ID NO: 42
AAGGGCTCGGGCTCGACCTCGGGCTCGGGCagcggtggcTCTAGAGGTTCTGGTGGCAG
CTCTGGAGGT
CIBN (truncated cryptochrome-interacting basic-helix-loop-helix protein)
for enhancing surface trafficking-
SEQ ID NO: 43
Atgaatggagctataggaggtgaccttttgctcaattttcctgacatgtcggtcctagagcgccaaagggctca
cctcaagtacctcaatcccacctttgattctcctctcgccggcttctttgccgattcttcaatgattaccggcg
gcgagatggacagctatctttcgactgccggtttgaatcttccgatgatgtacggtgagacgacggtggaaggt
gattcaagactctcaatttcgccggaaacgacgcttgggactggaaatttcaagGCaGCgaagtttgatacaga
gactaaggattgtaatgagGCgGCgaagaagatgacgatgaacagagatgacctagtagaagaaggagaagaag
agaagtcgaaaataacagagcaaaacaatgggagcacaaaaagcatcaagaagatgaaacacaaagccaagaaa
gaagagaacaatttctctaatgattcatctaaagtgacgaaggaattggagaaaacggattatattcat
Hemagglutinin (HA) epitope tag-
SEQ ID NO: 44
TACCCATACGATGTGCCAGATTACGCC
Stop codon-
SEQ ID NO: 45
tag

Cells were incubated for 24 h at 37° C. before further processing. HEK293T cells were imaged with 60× oil-immersion objective on the Nikon inverted confocal microscope. Shield-1 dissolved in complete growth media was added gently to the top of the media to 1 μM during imaging. The intensity profile was acquired with Nikon NIS-Elements analysis module and plotted by GraphPad Prism 7.

HEK 293T Cell Stimulation, Imaging, and Data Analysis for Shield-1 Induced Delocalization from the Plasma Membrane to the Cytosol

HEK 293T cells were plated in 24-well plates at 40% confluence and then transduced with 200 μL of transmembrane domain lentivirus supernatant and 200 μL of the mCherry-TEV protease lentivirus supernatant, and incubated for 48 h at 37° C. before further processing. Two extra wells without any infection are also plated for background subtraction. Shield-1 dissolved in complete growth media was added gently to the top of the media to 10 μM. The two non-infection wells are treated with shield-1 and without shield-1, respectively. HEK 293T cells were incubated for 18 h at 37° C. before imaging.

### HEK 293T Cell Stimulation, Imaging, and Data Analysis for Shield-1 Controlled Nuclear-Cytoplasmic Protein Distribution

HEK 293T cells were plated in 24-well plates at 40% confluence and then transduced with 50 μL of PKIt NES-EGFP-CapN-NLS-CapC lentivirus supernatant and 150 μL of NES-mCherry lentivirus supernatant. Cells were incubated for 24 h at 37° C. and replated in 24-well plates. Shield-I dissolved in complete growth media was added gently to the top of the media to 10 μM. HEK 293T cells were incubated for 18 h at 37° C. before imaging. HEK293T cells were imaged with 60× oil-immersion objective on the Nikon inverted confocal microscope. Individual cells and the nuclei are determined by mCherry signal. Mean intensity and area are acquired with Nikon NIS-Elements analysis module. Mean intensities were subtracted by background mean intensity, and the resulting number is multiplied with the area to give total intensities. The EGFP distribution ratio was calculated by the total intensity of EGFP in the cytosol to that in the whole cell. P value was determined by unpaired two-tailed t-tests.

### HEK 293T Cell Stimulation, Imaging, and Data Analysis for Shield-1 Dependent Gene Transcription Activation

HEK 293T cells were plated in 24-well plates at 40% confluence and then transduced with 100 μL of UAS-mCherry lentivirus supernatant, 100 μL of Gal4 DBD lentivirus supernatant, and 50 μL of VP16 lentivirus supernatant and incubated for 48 h at 37° C. before further processing. Two extra wells without any infection were also plated for background subtraction. Shield-1 dissolved in complete growth media was added gently to the top of the media to 10 μM. The two non-infection wells were treated with shield-1 and without shield-1 respectively. HEK 293T cells were incubated for 18 h at 37° C. before imaging. HEK 293T cells were imaged with 20× air objective on a Nikon inverted confocal microscope. Twelve fields of view were acquired for each condition. Mean intensity was acquired from each image with Nikon NIS-Elements analysis module. Mean intensities were subtracted by the average of the twelve background images' intensities and plotted by Prism 7. Several intensities were negative and were not shown in FIG. 12c, but they were counted in the average of each condition. P values were determined by unpaired two-tailed t-tests.

### AAV Supernatant Production

AAV supernatant was used for neuronal culture experiments. 6-well plate were pretreated with human fibronectin for 10 min at 37° C. HEK 293T cells were then plated in 6-well plates at 60-90% and transfected 2-3 h later. For each well, 0.35 μg viral DNA, 0.29 μg AAV1 serotype, 0.29 μg AAV2 serotype plasmid, and 0.7 μg helper plasmid pDF6 with 80 μL serum-free DMEM and 10 μL PEI max (PEI Max, pH 7.31 mg/mL, Polysciences) were mixed and incubated for 15 min at room temperature, and then 2 mL complete growth media was added and mixed. The DNA mix was added gently on the top of the cells. HEK293T cells were incubated for 40 h at 37° C. and then the supernatant (containing secreted AAV) was collected. The virus supernatant was stored in sterile Eppendorf tubes (0.5 mL/tube), flash frozen by liquid nitrogen and stored at −80° C.

### Concentrated AAV Production

AAV was prepared for in vivo use as described previously. Three T150 flasks of HEK 293T cells with fewer than 10 passages were transfected at 80% confluence. For each T150 flask, 5.2 μg construct plasmid, 4.35 μg AAV1 and 4.35 μg AAV2 serotype plasmids, 10.4 μg pDF6 adenovirus helper plasmid, and 130 μL PEI (PEI Max, pH 7.31 mg/mL, Polysciences) are mixed in 500 μL of serum-free DMEM for 10 min at room temperature. The DNA mixture was further suspended in 10 mL of complete media and added to cells. HEK293T cells were incubated for 40 to 48 h at 37° C. under 5% CO2. Cells are collected with a cell scraper, resuspended in 10 mL DPBS and then collected by centrifugation at 1200 rpm at room temperature for 5 min. The supernatant was discarded and the pellet was resuspended in 20 mL 100 mM NaCl, 20 mM Tris (pH=8.0). 1 mL of freshly prepared 10% sodium deoxycholate (Sigma-Aldrich) in water was added to the resuspended cells. Benzonase nuclease (Millipore-Sigma) was added to a final concentration of 50 units per mL. The solution was incubated in 37° C. water bath for 1 h and then centrifuged at 8000 rpm for 10 min. A heparin column was first equilibrated with 10 mL 100 mM NaCl, 20 mM Tris (pH=8.0) using a peristaltic pump, and then loaded with the virus supernatant. The column was washed with 25 mL of 100 mM NaCl, 20 mM Tris (pH=8.0), using the peristaltic pump, and then washed with 1 mL of 200 mM NaCl, 20 mM Tris (pH=8.0) and 1 mL of 300 mM NaCl, 20 mM Tris (pH=8.0) using a 5 mL syringe. For virus elution, 1.5 mL of 400 mM NaCl, 20 mM Tris (pH=8.0); 3.0 mL of 450 mM NaCl, 20 mM Tris (pH=8.0) and 1.5 mL of 500 mM NaCl, 20 mM Tris (pH=8.0) was applied sequentially to the column by a 5 mL syringe. The eluted virus was concentrated using Amicon Ultra 15 mL centrifugal units with a 100,000 molecular weight cut off at 8000 rpm for 6 min, to a final volume of 500 μL. For buffer exchange, 1 mL of sterile 20 mM Tris, 150 mM NaCl, 0.05% PF68 solution was added to the filter unit and the column was centrifuged again until the virus volume was ˜500 μL. The buffer exchange step was repeated 2 more times, and the final volume was ˜100 μL. The concentrated AAV was aliquoted in 5 μL to the 0.6 mL low retention microcentrifuge tubes (Thermo Fisher Scientific) and stored at −80° C.

### Neuronal Culture Experiment

Rat cortical neurons (Thermo Fisher Scientific, Cat #A1084001) were plated according to the user protocol. The half area 96-well glass plates (Corning, CLS4580-10EA) were coated with 50 μl 0.1 mg/ml of poly-D-lysine (Gibco) for 1 h, and then washed with ultrapure water twice. The frozen rat cortical neurons were quickly thawed in the 37° C. water bath until a small piece of ice was present. The cells were transferred to a 50 ml conical tube. To the cells, 1 ml pre-warmed 3:1 ratio of complete neurobasal media (NM) and glial enriching medium (GEM) mix was very slowly dropped in with gentle swirling. NM is composed of neurobasal (Thermo Fisher Scientific) supplemented 2% B27 (Thermo Fisher Scientific), 50 mM HEPES (Thermo Fisher Scientific), 1% Penicillin-Streptomycin (50 units/mL penicillin and 50 μg/mL streptomycin, Thermo Fisher Scientific), and 1% GlutaMAX (Thermo Fisher Scientific). GEM is composed of DMEM (Gibco) supplemented with 10% FBS (Fetal Bovine Serum, Sigma), 2% B27 (Thermo Fisher Scientific), 50 mM HEPES (Thermo Fisher Scientific), 1% Penicillin-Streptomycin (50 units/mL penicillin and 50 μg/mL streptomycin, Thermo Fisher Scientific), and 1% GlutaMAX (Thermo Fisher Scientific). An additional 2 ml of complete neurobasal media was added to the cells. The viable cell density was determined by adding 10 μL of the cell suspension to 10 μl 0.4% Trypan blue, followed by cell counting using hemocytometer. 0.25×10⁵ viable cells were plated on each well, and cells were grown at 37° C. with 5% CO₂. Half of the media was replaced with fresh complete neurobasal media within 4-24 h after plating. For maintaining the cells, half of the media was changed every three days.

For neuronal infection, either concentrated AAVes or supernatant AAVes were added the neurons at DIV5-DIV10 (days in vitro). Five days after infection, neurons were treated with 2 μM of shield-1 for 24 h and then imaged alive. Mean intensities were subtracted by the average of 5 background images' intensities and plotted by Prism 7. P values were determined by unpaired two-tailed t-tests.

### Animals

All procedures were carried out with approval from the University Committee on Use and Care of Animals at the University of Michigan. C57BL/6 mice were maintained under a 12-hour light/dark cycle and were provided with food and water ad libitum. Adult mice of both sexes were used.

Stereotactic Injection of AAV into the Mouse Brain

The stereotactic injection procedure was performed as previously described⁵⁴. Adult mice were anesthetized with isoflurane (5% for induction, 1.5% for maintenance), injected with 5 mg/kg of carprofen, and placed in a stereotactic apparatus. Body temperature was maintained at 35-37° C. 400 nL of concentrated AAV encoding shield-i-dependent gene regulation constructs under the hSyn promoter were stereotactically injected into the lateral hypothalamic area (±0.95 mm lateral to midline, −1.40 mm posterior, and −5.25 mm ventral to bregma) at a rate of 50 nL/min. The pipette was left undisturbed in the brain for 10 min following injection to allow for pressure to equalize and prevent a vacuum effect as the pipette was removed. Mice were given subcutaneous 1 mL saline injections and were recovered from surgery. Additional 5 mg/kg subcutaneous administration of carprofen was provided the day after surgery.

AAV Viral Injection into the Mouse Liver

Abdomen-shaved adult mice were placed on a stereotaxic apparatus to maintain anesthetic state under 1.5% isoflurane. The abdomen was disinfected by povidone followed by alcohol prior to surgery. A ˜2 cm midline incision was made in the abdomen to expose the liver. 1 μL of AAV encoding shield-i-dependent gene regulation constructs under the CMV promoter was delivered at a speed of 500 nL/min through a micropipette directly inserted into liver.

### Shield-1 Administration and Histology

For mouse brain, 7 days after the injection of the viral vectors, 1 μL of 1 mM aquashield-1 or saline control was locally administered into LHA by stereotactic injection. For mouse liver, 7 days after the expression of the viral vectors, animals were intraperitoneally injected with 2 doses of 40 mg/kg aquashield-1 or saline 24 h apart. 48 h after the first injection of aquashield-1 or saline, animals were euthanized and perfused with PBS and 4% paraformaldehyde (PFA).

Brain and liver tissues were harvested and fixed overnight in 4% PFA then cryoprotected in 30% sucrose for 48 h at 4° C. The fixed tissue was then embedded in optimum cutting temperature compound and sectioned at 30 m. Sections were rinsed in 0.1% PBS Tween-20 and stained with DAPI (1:10,000, Invitrogen, D1306) for 10 min at room temperature. Sections were then rinsed again and mounted with Prolong Gold mounting media (Invitrogen, P36930). Confocal images were taken on a Nikon A1 Confocal microscope.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

All publications, published patent documents, and patent applications cited herein are hereby incorporated by reference to the same extent as though each individual publication, published patent document, or patent application was specifically and individually indicated as being incorporated by reference.

### REFERENCES

(1) Song, H. K., and Eck, M. J. (2003) Structural basis of degradation signal recognition by SspB, a specificity-enhancing factor for the ClpXP proteolytic machine. Mol. Cell 12, 75-86.
(2) Yi, J. J., Wang, H., Vilela, M., Danuser, G., and Hahn, K. M. (2014) Manipulation of endogenous kinase activity in living cells using photoswitchable inhibitory peptides. ACS Synth. Biol. 3, 788-795.
(3) Matthew, A. N., Zephyr, J., Hill, C. J., Jahangir, M., Newton, A., Petropoulos, C. J., Huang, W., Kurt-Yilmaz, N., Schiffer, C. A., and Ali, A. (2017) Hepatitis C Virus NS3/4A Protease Inhibitors Incorporating Flexible P2 Quinoxalines Target Drug Resistant Viral Variants. J. Med. Chem. 60, 5699-5716.
(4) Dingwall C, and Laskey R A. (1991) Nuclear target sequences a consensus? Tibs 478-481.
(5) Xu, D., Farmer, A., Collett, G., Grishin, N. V., and Chook, Y. M. (2012) Sequence and structural analyses of nuclear export signals in the NESdb database. Mol. Biol. Cell (Weis, K., Ed.) 23, 3677-3693.
(6) Renicke, C., Schuster, D., Usherenko, S., Essen, L.-O., and Taxis, C. (2013) A LOV2 Domain-Based Optogenetic Tool to Control Protein Degradation and Cellular Function. Chem. Biol. 20, 619-626.
(7) Bonger, K. M., Chen, L. C., Liu, C. W., and Wandless, T. J. (2011) Small-molecule displacement of a cryptic degron causes conditional protein degradation. Nat. Chem. Biol. 7, 531-537.
(8) Wong, S., Mosabbir, A. A., and Truong, K. (2015) An engineered split intein for photoactivated protein trans-splicing. PLoS One 10, 1-16.
(9) So, W. H., Wong, C. T. T., and Xia, J. (2018) Peptide photocaging: A brief account of the chemistry and biological applications. Chinese Chem. Lett. 29, 1058-1062.
(10) Renner, C., and Moroder, L. (2006) Azobenzene as Conformational Switch in Model Peptides. ChemBioChem 7, 868-878.
(11) Lungu, O. I., Hallett, R. A., Choi, E. J., Aiken, M. J., Hahn, K. M., and Kuhlman, B. (2012) Designing Photoswitchable Peptides Using the AsLOV2 Domain. Chem. Biol. 19, 507-517.
(12) Bonger, K. M., Rakhit, R., Payumo, A. Y., Chen, J. K., and Wandless, T. J. (2014) General Method for Regulating Protein Stability with Light. ACS Chem. Biol. 9, 111-115.
(13) Niopek, D., Benzinger, D., Roensch, J., Draebing, T., Wehler, P., Eils, R., and Di Ventura, B. (2014) Engineering light-inducible nuclear localization signals for precise spatiotemporal control of protein dynamics in living cells. Nat. Commun. 5, 4404.
(14) Yumerefendi, H., Dickinson, D. J., Wang, H., Zimmerman, S. P., Bear, J. E., Goldstein, B., Hahn, K., and Kuhlman, B. (2015) Control of Protein Activity and Cell Fate Specification via Light-Mediated Nuclear Translocation. PLoS One (Palazzo, A. F., Ed.) 10, e0128443.
(15) Niopek, D., Wehler, P., Roensch, J., Eils, R., and Di Ventura, B. (2016) Optogenetic control of nuclear protein export. Nat. Commun. 7, 1-9.
(16) Guntas, G., Hallett, R. A., Zimmerman, S. P., Williams, T., Yumerefendi, H., Bear, J. E., and Kuhlman, B. (2015) Engineering an improved light-induced dimer (iLID) for controlling the localization and activity of signaling proteins. Proc. Natl. Acad. Sci. 112, 112-117.
(17) Konold, P. E., Mathes, T., Weienborn, J., Groot, M. L., Hegemann, P., and Kennis, J. T. M. (2016) Unfolding of the C-Terminal Ja Helix in the LOV2 Photoreceptor Domain Observed by Time-Resolved Vibrational Spectroscopy. J Phys. Chem. Lett. 7, 3472-3476.
(18) Peter, E., Dick, B., and Baeurle, S. A. (2010) Mechanism of signal transduction of the LOV2-Jα photosensor from Avena sativa. Nat. Commun. 1, 122-127.
(19) Ruggiero, E., Alonso-de Castro, S., Habtemariam, A., and Salassa, L. (2016) Upconverting nanoparticles for the near infrared photoactivation of transition metal complexes: new opportunities and challenges in medicinal inorganic photochemistry. Dalt. Trans. 45, 13012-13020.
(20) Stanton, B. Z., Chory, E. J., and Crabtree, G. R. (2018) Chemically induced proximity in biology and medicine. Science (80-.). 359, eaao5902.
(21) Wu, H. D., Kikuchi, M., Dagliyan, O., Aragaki, A. K., Nakamura, H., Dokholyan, N. V., Umehara, T., and Inoue, T. (2020) Rational design and implementation of a chemically inducible heterotrimerization system. Nat. Methods 17, 928-936.
(22) Karginov, A. V., Ding, F., Kota, P., Dokholyan, N. V., and Hahn, K. M. (2010) Engineered allosteric activation of kinases in living cells. Nat. Biotechnol. 28, 743-747.
(23) Farrants, H., Tarnawski, M., Müller, T. G., Otsuka, S., Hiblot, J., Koch, B., Kueblbeck, M., Krausslich, H.-G., Ellenberg, J., and Johnsson, K. (2020) Chemogenetic Control of Nanobodies. Nat. Methods 17, 279-282.
(24) Clackson, T., Yang, W., Rozamus, L. W., Hatada, M., Amara, J. F., Rollins, C. T., Stevenson, L. F., Magari, S. R., Wood, S. A., Courage, N. L., Lu, X., Cerasoli, F., Gilman, M., and Holt, D. A. (1998) Redesigning an FKBP-ligand interface to generate chemical dimerizers with novel specificity. Proc. Natl. Acad. Sci. U.S.A 95, 10437-10442.
(25) Banaszynski, L. A., Sellmyer, M. A., Contag, C. H., Wandless, T. J., and Thorne, S. H. (2008) Chemical control of protein stability and function in living mice. Nat. Med. 14, 1123-1127.
(26) Kapust, R. B., Tözsér, J., Copeland, T. D., and Waugh, D. S. (2002) The P1′ specificity of tobacco etch virus protease. Biochem. Biophys. Res. Commun. 294, 949-955.
(27) Guntas, G., Hallett, R. A., Zimmerman, S. P., Williams, T., Yumerefendi, H., Bear, J. E., and Kuhlman, B. (2015) Engineering an improved light-induced dimer (iLID) for controlling the localization and activity of signaling proteins. Proc. Natl. Acad. Sci. U.S.A
(28) Castellano, F., Montcourrier, P., and Chavrier, P. (2000) Membrane recruitment of Rac1 triggers phagocytosis. J Cell Sci. 113, 2955-2961.
(29) Pham, E., Mills, E., and Truong, K. (2011) A synthetic photoactivated protein to generate local or global Ca 2+ signals. Chem. Biol. 18, 880-890.
(30) Nguyen, T., Pappireddi, N., and Wuhr, M. (2019) Proteomics of nucleocytoplasmic partitioning. Curr. Opin. Chem. Biol. 48, 55-63.
(31) Ptashne, M., and Gann, A. (1997) Transcriptional activation by recruitment. Nature 386, 569-577.
(32) Strickland, D., Lin, Y., Wagner, E., Hope, C. M., Zayner, J., Antoniou, C., Sosnick, T. R., Weiss, E. L., and Glotzer, M. (2012) TULIPs: Tunable, light-controlled interacting protein tags for cell biology. Nat. Methods 9, 379-384.
(33) Wu, Y. I., Frey, D., Lungu, O. I., Jaehrig, A., Schlichting, I., Kuhlman, B., and Hahn, K. M. (2009) A genetically encoded photoactivatable Rac controls the motility of living cells. Nature 461, 104-108.
(34) Baarlink, C., Wang, H., and Grosse, R. (2013) Nuclear Actin Network Assembly by Formins Regulates the SRF Coactivator MAL. Science (80-.). 340, 864-867.
(35) Cosentino, C., Alberio, L., Gazzarrini, S., Aquila, M., Romano, E., Cermenati, S., Zuccolini, P., Petersen, J., Beltrame, M., Van Etten, J. L., Christie, J. M., Thiel, G., and Moroni, A. (2015) Engineering of a light-gated potassium channel. Science (80-.). 348, 707-710.
(36) McCauley, J. A., and Rudd, M. T. (2016) Hepatitis C virus NS3/4a protease inhibitors. Curr. Opin. Pharmacol. 30, 84-92.
(37) Cunningham-Bryant, D., Dieter, E. M., Foight, G. W., Rose, J. C., Loutey, D. E., and Maly, D. J. (2019) A Chemically Disrupted Proximity System for Controlling Dynamic Cellular Processes. J. Am. Chem. Soc. 141, 3352-3355.
(38) Rose, J. C., Huang, P. S., Camp, N. D., Ye, J., Leidal, A. M., Goreshnik, I., Trevillian, B. M., Dickinson, M. S., Cunningham-Bryant, D., Debnath, J., Baker, D., Wolf-Yadlin, A., and Maly, D. J. (2017) A computationally engineered RAS rheostat reveals RAS-ERK signaling dynamics. Nat. Chem. Biol. 13, 119-126.
(39) Wang, W., Wildes, C. P., Pattarabanjird, T., Sanchez, M. I., Glober, G. F., Matthews, G. A., Tye, K. M., and Ting, A. Y. (2017) A light- and calcium-gated transcription factor for imaging and manipulating activated neurons. Nat. Biotechnol. 35, 864-871.
(40) Lam, S. S., Martell, J. D., Kamer, K. J., Deerinck, T. J., Ellisman, M. H., Mootha, V. K., and Ting, A. Y. (2015) Directed evolution of APEX2 for electron microscopy and proximity labeling. Nat. Methods 12, 51-54.
(41) Abraham, M. J., Murtola, T., Schulz, R., Pall, S., Smith, J. C., Hess, B., and Lindah, E. (2015) Gromacs: High performance molecular simulations through multi-level parallelism from laptops to supercomputers. SoftwareX.
(42) Lindorff-Larsen, K., Piana, S., Palmo, K., Maragakis, P., Klepeis, J. L., Dror, R. O., and Shaw, D. E. (2010) Improved side-chain torsion potentials for the Amber ff99SB protein force field. Proteins Struct. Funct. Bioinforma.
(43) Jorgensen, W. L., Chandrasekhar, J., Madura, J. D., Impey, R. W., and Klein, M. L. (1983) Comparison of simple potential functions for simulating liquid water. J. Chem. Phys.
(44) Guex, N. and Peitsch, M. C. (1997) Swiss PDB Viewer—References. Electrophoresis.
(45) Krivov, G. G., Shapovalov, M. V., and Dunbrack, R. L. (2009) Improved prediction of protein side-chain conformations with SCWRL4. Proteins Struct. Funct. Bioinforma. 77, 778-795.
(46) Darden, T., York, D., and Pedersen, L. (1993) Particle mesh Ewald: An N·log(N) method for Ewald sums in large systems. J Chem. Phys. 98, 10089-10092.
(47) Hess, B., Bekker, H., Berendsen, H. J. C., and Fraaije, J. G. E. M. (1997) LINCS: A linear constraint solver for molecular simulations. J. Comput. Chem. 18, 1463-1472.
(48) Miyamoto, S., and Kollman, P. A. (1992) Settle: An analytical version of the SHAKE and RATTLE algorithm for rigid water models. J Comput. Chem. 13, 952-962.
(49) Berendsen, H. J. C., Postma, J. P. M., van Gunsteren, W. F., DiNola, A., and Haak, J. R. (1984) Molecular dynamics with coupling to an external bath. J. Chem. Phys. 81, 3684-3690.
(50) Nose, S. (1984) A molecular dynamics method for simulations in the canonical ensemble. Mol. Phys. 52, 255-268.
(51) Hoover, W. G. (1985) Canonical dynamics: Equilibrium phase-space distributions. Phys. Rev. A.
(52) Parrinello, M., and Rahman, A. (1981) Polymorphic transitions in single crystals: A new molecular dynamics method. J. Appl. Phys. 52, 7182-7190.
(53) Daura, X., Gademann, K., Jaun, B., Seebach, D., van Gunsteren, W. F., and Mark, A. E. (1999) Peptide Folding: When Simulation Meets Experiment. Angew. Chemie Int. Ed. 38, 236-240.
(54) Li, P., Li, S.-B., Wang, X., Phillips, C. D., Schwarz, L. A., Luo, L., de Lecea, L., and Krasnow, M. A. (2020) Brain Circuit of Claustrophobia-like Behavior in Mice Identified by Upstream Tracing of Sighing. Cell Rep. 31, 107779.

We claim:

1. A method of regulating the activity of a peptide, comprising:
a) generating a nucleic acid construct, comprising:
1) a nucleic acid that encodes at least one chemically-activated protein domain (CAP) comprising a ligand binding site; and
2) a nucleic acid that encodes a peptide;
b) administering said nucleic acid construct to one or more cells to generate a fusion protein wherein said fusion protein comprises said at least one CAP linked to said peptide; and
c) administering a ligand to said one or more cells wherein said ligand binds to said ligand binding site of said CAP of said fusion protein, wherein said administering of said ligand increases said activity of said peptide.

2. The method of claim 1, wherein said at least one CAP comprises CapN that binds to the N-terminus of said peptide, and/or CapC that binds to the C-terminus of said peptide.

3. The method of claim 1, wherein said administering said nucleic acid construct comprises direct injection of said nucleic acid construct, macromolecule-mediated liposomal and/or biopolymer gene delivery, plasmid delivery, and/or viral delivery.

4. The method of claim 3, wherein said viral delivery is selected from the group consisting of adeno-associated viral (AAV) delivery, adenoviral delivery, lentivirus delivery, vaccina virus delivery and retroviral delivery.

5. The method of claim 1, wherein said nucleic acid construct is stably expressed or transiently expressed.

6. The method of claim 5, wherein said expression is intracellular expression or extracellular expression.

7. The method of claim 1, wherein said ligand binding site comprises a FKBP binding domain.

8. The method of claim 1, wherein said peptide is SsrA, a nuclear localization signal peptide (NLS), a nuclear export signal peptide, met-enkephalin, or TEV protease cleavage site.

9. The method of claim 1, wherein said peptide is an enzyme activation peptide, an enzyme inhibition peptide, an enzyme regulation peptide, a binding peptide, a localization peptide, or a degradation peptide.

10. The method of claim 1, wherein said ligand is shield-I and/or aquashield-1.

11. The method of claim 1, wherein said administering is parenteral administering and/or non-parenteral administering.

12. The method of claim 1, wherein said one or more cells are cells in vitro or cells in vivo.

13. The method of claim 12, wherein said cells in vivo are neuronal cells or liver cells.

14. The method of claim 1, wherein said nucleic acid construct comprises at least one nucleic acid selected from the group encoding Aga2p, a reporter gene, TetR DBD, SspB, Vp16, P2A, a linker, FLAG, UAS-mCherry and an internal ribosome entry site (IRES).

15. The method of claim 1, wherein said nucleic acid construct further comprises a promoter nucleic acid selected from the group consisting of CMV, CAG and synapsin.

16. The method of claim 1, comprising a protein of interest (POI).

17. The method of claim 16, wherein said POI is a transcription factor, a kinase, a gene editing enzyme, or a label.

18. The method of claim 16, wherein said POI is evolved green fluorescent protein (EGFP).

19. The method of claim 1, further comprising measuring the activity of said peptide wherein said measuring comprises measuring the localization, structure and/or function of said peptide.

20. A kit, comprising:
a) a nucleic acid construct, comprising:
1) a nucleic acid that encodes at least one chemically-activated protein domain (CAP) comprising a ligand binding site; and
2) a nucleic acid that encodes a peptide;
c) a gene transfer reagent and/or vector; and
d) ligand that binds to said ligand binding site.

21. The kit of claim 20, comprising one or more control ligands.

22. A composition, comprising:
a) a nucleic acid construct, comprising:
1) a nucleic acid that encodes at least one chemically-activated protein domain (CAP) comprising a ligand binding site wherein said ligand is shield-1 and/or aquashield-1;
2) a nucleic acid that encodes a peptide; and
C) a gene transfer reagent and/or vector.

23. A fusion protein, comprising
a) a protein expressed by a nucleic acid that encodes at least one chemically-activated protein domain (CAP) comprising a ligand binding site wherein said ligand is shield-1 and/or aquashield-1; and
b) a peptide expressed by a nucleic acid wherein said peptide is regulated by said shield-1 and/or aquashield-1.
